(19) United States
(12) Patent Application Publication
(10) Pub. No.: US 2008/0195327 A1
(43) Pub. Date: Aug. 14, 2008

(54) Methods of Analysis of Polymorphisms and Uses Thereof

(75) Inventor: Robert Peter Young, Parnell (NZ)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Synergenz Bioscience Limited, Tortola (VG)

(21) Appl. No.: 11/874,185

(22) Filed: Oct. 17, 2007

(30) Foreign Application Priority Data
Oct. 17, 2006 (NZ) NZ 550643
Nov. 22, 2006 (NZ) NZ 551534
Dec. 7, 2006 (NZ) NZ 551883
Apr. 23, 2007 (NZ) NZ 554707
Jul. 31, 2007 (NZ) NZ 560262
Jul. 31, 2007 (NZ) NZ 560263

(51) Int. Cl. G01N 33/48

(52) U.S. Cl. 702/20

(57) ABSTRACT

The present invention provides methods for the assessment of a subject's suitability for an intervention in respect of one or more diseases. The methods are dependant on the results of at least one genetic analysis, in particular genetic analyses that are predictive of predisposition to one or more diseases, including one or more genetic analyses of genetic polymorphisms associated with one or more diseases.

### RELATED APPLICATIONS

This application claims priority to New Zealand Patent Application Nos. 550643, filed Oct. 17, 2006; 551534, filed Nov. 22, 2006; 551883, filed Dec. 7, 2006; 554707, filed Apr. 23, 2007; 560262, filed Jul. 31, 2007; and 560263, filed Jul. 31, 2007, all of which are incorporated herein by reference in their entireties.

### FIELD OF THE INVENTION

The present invention is concerned with methods of assessing diseases that result from the combined or interactive effects of two or more genetic variants, and methods of and systems for assessing subject data (including genetic data) indicative of predisposition to various diseases or conditions, and in particular for assessing a subject's suitability for an intervention using an analysis of genetic polymorphisms.

### BACKGROUND OF THE INVENTION

It has been estimated that over 4500 identified human diseases or conditions are due to genetic defects. Diseases with a direct genetic cause, such as, for example, sickle cell anaemia, can be straightforward to diagnose or predict on the basis of genetic analysis. For example, the identification in the genome of a subject of an autosomal dominant genetic defect known to cause a disease means that that subject will, barring an intervening action, manifest that disease. Importantly, it is becoming increasingly apparent that a great proportion of diseases or conditions have a genetic component, whereby a subject's particular genetic makeup can for example render the subject more or less susceptible to a given disease or condition, or can ameliorate or exacerbate the symptoms of a disease or condition suffered by the subject. Often in such diseases the genetic component is multivariate, complex, and refractory to simple understanding.

Diseases that result from the combined or interactive effects of two or more genetic variants, with or without environmental factors, are called complex diseases and include cancer, coronary artery disease, diabetes, stroke, and chronic obstructive pulmonary disease (COPD). Although combining non-genetic risk factors to determine a risk level of outcome has been in applied to coronary artery disease, (by combining individual factors such as blood pressure, gender, fasting cholesterol, and smoking status), there are no such methods in combining the effects of multiple genetic factors with non-genetic factors. There is a growing realization that the complex diseases, for which examples are given above, can result from the combined effects of common genetic variants or polymorphisms rather than mutations which are rare (believed to be present in less than 1% of the general population). Moreover, these relatively common polymorphisms can confer either susceptibility and/or protective effects on the development of these diseases. In addition, the likelihood that these polymorphisms are actually expressed (termed penetrance) as a disease or clinical manifestation requires a quantum of environmental exposure before such a genetic tendency can be clinically detected.

Clearly, the ability to predict susceptibility to one or more diseases or conditions is of great significance. Subjects, informed of their susceptibility to one or more diseases or conditions and whether found to be of greater or lesser susceptibility to a given disease, would be better able to determine an appropriate lifestyle and better able to manage their health. Health care providers would be better able to manage health care plans that could be targeted to the needs of individual subjects.

There is thus a need for a method for assessing a subject's risk of developing a disease using genetic (and optionally non-genetic) risk factors so as to assess the subject's suitability for undergoing a medical intervention.

It is an object of the present invention to go some way towards meeting this need and/or to provide the public with a useful choice.

### BRIEF DESCRIPTION OF THE INVENTION

The Applicant's recent studies have identified a number of genetic variants or polymorphisms that confer susceptibility to protection from COPD, occupational COPD (OCOPD), lung cancer, and acute coronary syndrome (ACS). The biological basis of just how these polymorphisms interact or combine to determine risk remains unclear.

The Applicants have found that an assessment approach which determines a subject's net score following the balancing of the number of polymorphisms associated with protection from a disease against the number of polymorphisms associated with susceptibility to that disease present in the subject is indicative of that subject's suitability for a medical intervention. Furthermore, the applicants have determined that this approach is widely applicable, on a disease-by-disease basis.

It is broadly to this approach to risk assessment that the present invention is directed.

Accordingly, in a first aspect, an embodiment of the present invention provides a method of assessing a subject's suitability for an intervention that is diagnostic of or therapeutic for a disease, the method including:

a) providing a net score for said subject, wherein the net score is or has been determined by:
i) providing the result of one or more genetic tests of a sample from the subject, and analysing the result for the presence or absence of protective polymorphisms and for the presence or absence of susceptibility polymorphisms, wherein said protective and susceptibility polymorphisms are associated with said disease,
ii) assigning a positive score for each protective polymorphism and a negative score for each susceptibility polymorphism or vice versa;
iii) calculating a net score for said subject by representing the balance between the combined value of the protective polymorphisms and the combined value of the susceptibility polymorphisms present in the subject sample;

b) providing a distribution of net scores for disease sufferers and non-sufferers wherein the net scores for disease sufferers and non-sufferers are or have been determined in the same manner as the net score determined for said subject; and

c) determining whether the net score for said subject lies within a threshold on said distribution separating individuals deemed suitable for said intervention from those for whom said intervention is deemed unsuitable;

wherein a net score within said threshold can be indicative of the subject's suitability for the intervention, and wherein a net score outside the threshold can be indicative of the subject's unsuitability for the intervention.

The value assigned to each protective polymorphism can be the same or can be different. The value assigned to each susceptibility polymorphism can be the same or can be different, with either each protective polymorphism having a negative value and each susceptibility polymorphism having a positive value, or vice versa.

In one embodiment, the intervention can be a diagnostic test for said disease.

In another embodiment, the intervention can be a therapy for said disease, more preferably a preventative therapy for said disease.

Preferably, the disease can be lung cancer, more preferably the disease can be lung cancer and the protective and susceptibility polymorphisms can be selected from the group including:
the −133 G/C polymorphism in the Interleukin-18 gene;
the −1053 C/T polymorphism in the CYP 2E1 gene;
the Arg197gln polymorphism in the Nat2 gene;
the −511 G/A polymorphism in the Interleukin 1B gene;
the Ala 9 Thr polymorphism in the Anti-chymotrypsin gene;
the S allele polymorphism in the Alpha1-antitrypsin gene;
the −251 A/T polymorphism in the Interleukin-8 gene;
the Lys 751 gln polymorphism in the XPD gene;
the +760 G/C polymorphism in the SOD3 gene;
the Phe257Ser polymorphism in the REV gene;
the Z allele polymorphism in the Alpha1-antitrypsin gene;
the R19W A/G polymorphism in the Cerberus 1 (Cer 1) gene;
the Ser 307Ser G/T polymorphism in the X-ray repair complementing defective repair in Chinese hamster cells 4 gene (XRCC4);
the K3326X A/T polymorphism in the breast cancer 2 early onset gene (BRCA2) gene;
the V433M A/G polymorphism in the Integrin alpha-11 gene;
the E375G T/C polymorphism the gene encoding Calcium/calmodulin-dependent protein kinase 1 (CAMKK1);
the A/T c74delA polymorphism in the gene encoding cytochrome P450 polypeptide CYP3A43 (CYP3A43);
the A/C (rs2279115) polymorphism in the gene encoding B-cell CLL/lymphoma 2 (BCL2);
the A/G at +3100 in the 3′UTR (rs2317676) polymorphism of the gene encoding Integrin beta 3 (ITGB3);
the −3714 G/T (rs6413429) polymorphism in the gene encoding Dopamine transporter 1 (DAT1);
the A/G (rs139417) polymorphism in the gene encoding Tumor necrosis factor receptor 1 (TNFR1);
the C/Del (rs1799732) polymorphism in the gene encoding Dopamine receptor D2 (DRD2);
the C/T (rs763110) polymorphism in the gene encoding Fas ligand (FasL); or C/T (rs5743836) polymorphism in the gene encoding Toll-like receptor 9 (TLR9);
the −81 C/T (rs2273953) polymorphism in the 5′ UTR of the gene encoding Tumor protein P73 (TP73);

or one or more polymorphisms in linkage disequilibrium with one or more of said polymorphisms.

More preferably, said intervention can be a CT scan for lung cancer.

When the disease is a lung disease, the protective polymorphisms analysed can be selected from one or more of the group including:
+760GG or +760CG within the gene encoding superoxide dismutase 3 (SOD3);
−1296TT within the promoter of the gene encoding tissue inhibitor of metalloproteinase 3 (TIMP3);
CC (homozygous P allele) within codon 10 of the gene encoding transforming growth factor beta (TGFβ);
2G2G within the promoter of the gene encoding metalloproteinase 1 (MMP1);
or one or more polymorphisms in linkage disequilibrium with one or more of these polymorphisms.

Linkage disequilibrium is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204).

Preferably, the susceptibility polymorphisms analyzed can be selected from one or more of the group including:
−82AA within the promoter of the gene encoding human macrophage elastase (MMP12);
−1562CT or −1562TT within the promoter of the gene encoding metalloproteinase 9 (MMP9);
1237AG or 1237AA (Tt or tt allele genotypes) within the 3′ region of the gene encoding α1-antitrypsin (α1AT); or
one or more polymorphisms in linkage disequilibrium with one or more of these polymorphisms.

When the disease is COPD, the protective polymorphisms analysed can be selected from one or more of the group including:
−765 CC or CG in the promoter of the gene encoding cyclooxygenase 2 (COX2);
Arg 130 Gln AA in the gene encoding Interleukin-13 (IL-13);
Asp 298 Glu TT in the gene encoding nitric oxide synthase 3 (NOS3);
Lys 420 Thr AA or AC in the gene encoding vitamin binding protein (VDBP);
Glu 416 Asp TT or TG in the gene encoding VDBP;
Ile 105 Val AA in the gene encoding glutathione S-transferase (GSTP1);
MS in the gene encoding α1-antitrypsin (α1AT);
the +489 GG genotype in the gene encoding Tissue Necrosis factor α (TNFα);
the −308 GG genotype in the gene encoding TNFα;
the C89Y AA or AG genotype in the gene encoding SMAD3;
the 161 GG genotype in the gene encoding Mannose binding lectin 2 (MBL2);
the −1903 AA genotype in the gene encoding Chymase 1 (CMA1);
the Arg 197 Gln AA genotype in the gene encoding N-Acetyl transferase 2 (NAT2);
the His 139 Arg GG genotype in the gene encoding Microsomal epoxide hydrolase (MEH);
the −366 AA or AG genotype in the gene encoding 5 Lipo-oxygenase (ALOX5);
the HOM T2437C TT genotype in the gene encoding Heat Shock Protein 70 (HSP 70);
the exon 1+49 CT or TT genotype in the gene encoding Elafin;
the Gln 27 Glu GG genotype in the gene encoding P2 Adrenergic receptor (ADBR);
the −1607 1G1G or 1G2G genotype in the promoter of the gene encoding Matrix Metalloproteinase 1 (MMP1);

or one or more polymorphisms in linkage disequilibrium with one or more of these polymorphisms.

Preferably, the susceptibility polymorphisms analyzed can be selected from one or more of the group including:
Arg 16 Gly GG in the gene encoding P2-adrenoreceptor (ADRB2);
105 AA in the gene encoding Interleukin-18 (IL-18);
−133 CC in the promoter of the gene encoding IL-18;
−675 5G5G in the promoter of the gene encoding plasminogen activator inhibitor 1 (PAI-1);
−1055 TT in the promoter of the gene encoding IL-13;
874 TT in the gene encoding interferon gamma (IFNγ);
the +489 AA or AG genotype in the gene encoding TNFα;
the −308 AA or AG genotype in the gene encoding TNFα;
the C89Y GG genotype in the gene encoding SMAD3;
the E469K GG genotype in the gene encoding Intracellular Adhesion molecule 1 (ICAM1);
the Gly 881 Arg GC or CC genotype in the gene encoding Caspase (NOD2);
the −511 GG genotype in the gene encoding IL1B;
the Tyr 113 His TT genotype in the gene encoding MEH;
the −366 GG genotype in the gene encoding ALOX5;
the HOM T2437C CC or CT genotype in the gene encoding HSP 70;
the +13924 AA genotype in the gene encoding Chloride Channel Calcium-activated 1 (CLCA1);
the −159 CC genotype in the gene encoding Monocyte differentiation antigen CD-14 (CD-14);
or one or more polymorphisms in linkage disequilibrium with one or more of these polymorphisms.

When the disease is OCOPD, the protective polymorphisms analysed can be selected from one or more of the group including:
−765 CC or CG in the promoter of the gene encoding COX2;
−251 AA in the promoter of the gene encoding interleukin-8 (IL-8);
Lys 420 Thr AA in the gene encoding VDBP;
Glu 416 Asp TT or TG in the gene encoding VDBP;
exon 3 T/C RR in the gene encoding microsomal epoxide hydrolase (MEH);
Arg 312 Gln AG or GG in the gene encoding SOD3;
MS or SS in the gene encoding α1AT;
Asp 299 Gly AG or GG in the gene encoding toll-like receptor 4 (TLR4);
Gln 27 Glu CC in the gene encoding ADRB2;
−518 AA in the gene encoding IL-11;
Asp 298 Glu TT in the gene encoding NOS3; or
one or more polymorphisms in linkage disequilibrium with one or more of these polymorphisms.

Preferably, the susceptibility polymorphisms analysed can be selected from one or more of the group including:
−765 GG in the promoter of the gene encoding COX2;
105 AA in the gene encoding IL-18;
−133 CC in the promoter of the gene encoding IL-18;
−675 5G5G in the promoter of the gene encoding PAI-1;
Lys 420 Thr CC in the gene encoding VDBP;
Glu 416 Asp GG in the gene encoding VDBP;
Ile 105 Val GG in the gene encoding GSTP1;
Arg 312 Gln AA in the gene encoding SOD3;
−1055 TT in the promoter of the gene encoding IL-13;
3′ 1237 Tt or tt in the gene encoding α1AT;
−1607 2G2G in the promoter of the gene encoding MMP1; or
one or more polymorphisms in linkage disequilibrium with one or more of these polymorphisms.

When the disease is lung cancer, the protective polymorphisms analysed can be selected from one or more of the group including:
the Asp 298 Glu TT genotype in the gene encoding NOS3;
the Arg 312 Gln CG or GG genotype in the gene encoding SOD3;
the Asn 357 Ser AG or GG genotype in the gene encoding MMP12;
the 105 AC or CC genotype in the gene encoding IL-18;
the −133 CG or GG genotype in the gene encoding IL-18;
the −765 CC or CG genotype in the promoter of the gene encoding COX2;
the −221 TT genotype in the gene encoding Mucin 5AC (MUC5AC);
the intron 1 C/T TT genotype in the gene encoding Arginase 1 (Arg1);
the Leu252Val GG genotype in the gene encoding Insulin-like growth factor II receptor (IGF2R);
the −1082 GG genotype in the gene encoding Interleukin 10 (IL-10);
the −251 AA genotype in the gene encoding Interleukin 8 (IL-8);
the Arg 399 Gln AA genotype in the X-ray repair complementing defective in Chinese hamster 1 (XRCC1) gene;
the A870G GG genotype in the gene encoding cyclin D (CCND1);
the −751 GG genotype in the promoter of the xeroderma pigmentosum complementation group D (XPD) gene;
the Ile 462 Val AG or GG genotype in the gene encoding cytochrome P450 1A1 (CYP1A1);
the Ser 326 Cys GG genotype in the gene encoding 8-Oxoguanine DNA glycolase (OGG1);
the Phe 257 Ser CC genotype in the gene encoding REV1;
the E375G T/C TT genotype in the gene encoding CAMKK1;
the −81 C/T (rs2273953) CC genotype the gene encoding TP73;
the A/C (rs2279115) AA genotype in the gene encoding BCL2;
the +3100 A/G (rs2317676) AG or GG genotype in the gene encoding ITGB3;
the C/Del (rs1799732) CDel or DelDel genotype in the gene encoding DRD2; or
the C/T (rs763110) TT genotype in the gene encoding FasL;
or one or more polymorphisms in linkage disequilibrium with any one or more of these polymorphisms.

Preferably, the susceptibility polymorphisms analyzed can be selected from one or more of the group including:
the −786 TT genotype in the promoter of the gene encoding NOS3;
the Ala 15 Thr GG genotype in the gene encoding anti-chymotrypsin (ACT);
the 105 AA genotype in the gene encoding IL-18;
the −133 CC genotype in the promoter of the gene encoding IL-18;
the 874 AA genotype in the gene encoding IFNγ;
the −765 GG genotype in the promoter of the gene encoding COX2;
the −447 CC or GC genotype in the gene encoding Connective tissue growth factor (CTGF); and
the +161 AA or AG genotype in the gene encoding MBL2.
the −511 GG genotype in the gene encoding IL-1B;
the A-670G AA genotype in the gene encoding FAS (Apo-1/CD95);
the Arg 197 Gln GG genotype in the gene encoding N-acetyltransferase 2 (NAT2);
the Ile462 Val AA genotype in the gene encoding CYP1A1;
the 1019 G/C Pst I CC or CG genotype in the gene encoding cytochrome P450 2E1 (CYP2E1);
the C/T Rsa I TT or TC genotype in the gene encoding CYP2E1;
the GSTM null genotype in the gene encoding GSTM;
the −1607 2G/2G genotype in the promoter of the gene encoding MMP1;
the Gln 185 Glu CC genotype in the gene encoding Nibrin (NBS1);
the Asp 148 Glu GG genotype in the gene encoding Apex nuclease (APE1);
the R19W A/G AA or GG genotype in the gene encoding Cer 1;
the Ser307Ser G/T GG or GT genotype in the XRCC4 gene;
the K3326X A/T AT or TT genotype in the BRCA2 gene;
the V433M A/G AA genotype in the gene encoding Integrin alpha-11;
the A/T c74delA AT or TT genotype in the gene encoding CYP3A43;
the −3714 G/T (rs6413429) GT or TT genotype in the gene encoding DAT1;
the A/G (rs1139417) AA genotype in the gene encoding TNFR1; or
the C/T (rs5743836) CC genotype in the gene encoding TLR9;
or one or more polymorphisms in linkage disequilibrium with any one or more of these polymorphisms.

When the disease is ACS, the protective polymorphisms analysed can be selected from one or more of the group including:
the Ser52Ser (223 C/T) CC genotype in the gene encoding FGF2;
the Q576R A/G AA genotype in the gene encoding IL4RA;
the Thr26Asn A/C CC genotype in the gene encoding LTA;
the Hom T2437C CC or CT genotype in the gene encoding HSP70;
the Asp299Gly A/G AG or GG genotype in the gene encoding TLR4;
the Thr399Ile C/T CT or TT genotype in the gene encoding TLR4;
the 874 A/T TT genotype in the gene encoding IFNG;
the −63 T/A AA genotype in the gene encoding NFKBIL1;
the −1630 Ins/Del (AACTT/Del) Ins/Del or Del/Del genotype in the gene encoding PDGFRA;
the −589 C/T CT or TT genotype in the gene encoding IL-4;
the −588 C/T CC genotype in the gene encoding GCLM;
the −1084 A/G GG genotype in the gene encoding IL-10;
the K469E A/G AA genotype in the gene encoding ICAM1;
the −23 C/G GG genotype in the gene encoding BAT1;
the Glu298Asp G/T GG genotype in the gene encoding NOS3;
the Arg213Gly C/G CG or GG genotype in the gene encoding SOD3;
the −668 4G/5G 5G5G genotype in the gene encoding PAI-1; or
the −181 A/G GG genotype in the gene encoding MMP7;
or one or more polymorphisms in linkage disequilibrium with any one or more of these polymorphisms.

Preferably, the susceptibility polymorphisms analyzed can be selected from one or more of the group including:
the −1903 A/G GG genotype in the gene encoding CMA1;
the −509 C/T CC genotype in the gene encoding TGFB1;
the −82 A/G GG genotype in the gene encoding MMP12;
the Ser52Ser (223 C/T) CT or TT genotype in the gene encoding FGF2;
the Q576R A/G GG genotype in the gene encoding IL4RA;
the Hom T2437C TT genotype in the gene encoding HSP70;
the Asp299Gly A/G AA genotype in the gene encoding TLR4;
the Thr399Ile C/T CC genotype in the gene encoding TLR4;
the −1630 Ins/Del (AACTT/Del) Ins Ins (AACTT AACTT) genotype in the gene encoding PDGFRA;
the −589 C/T CC genotype in the gene encoding IL4;
the −1607 1G/2G (Del/G) Del Del (1G 1G) genotype in the gene encoding MMP1;
the 12 IN5 C/T TT genotype in the gene encoding PDGFA;
the −588 C/T CT or TT genotype in the gene encoding GCLM;
the Ile132Val A/G AA genotype in the gene encoding OR13G1;
the Glu288Val A/T (M/S) AT or TT (MS or SS) genotype in the gene encoding α1-AT; or
the +459 C/T Intron 1 CT or TT genotype in the gene encoding MIP1A;
or one or more polymorphisms in linkage disequilibrium with any one or more of these polymorphisms.

Preferably, all polymorphisms of the group are analysed.

In one embodiment each protective polymorphism can be assigned a value of −1 and each susceptibility polymorphism can be assigned a value of +1.

In one embodiment each protective polymorphism can be assigned a value of +1 and each susceptibility polymorphism can be assigned a value of −1.

In various embodiments the subject can be or has been a smoker.

Preferably, the methods of the invention can be performed in conjunction with an analysis of one or more risk factors, including one or more epidemiological risk factors, associated with the risk of developing a lung disease including COPD, emphysema, OCOPD, and lung cancer. Such epidemiological risk factors include but are not limited to smoking or exposure to tobacco smoke, age, sex, and familial history.

In another embodiment, the present invention can provide a kit for assessing a subject's suitability for an intervention diagnostic of or therapeutic for a disease, said kit including a means of analysing a sample from said subject for the presence or absence of one or more protective polymorphisms and one or more susceptibility polymorphisms as described herein.

In yet a further embodiment, the present invention can provide a method of diagnostic, prophylactic or therapeutic treatment of a disease in a subject whose suitability for said treatment is or has been determined by a method as defined above which includes the steps of communicating to said subject said net susceptibility score, and advising on changes to the subject's lifestyle that could reduce the risk of developing said disease.

In still a further embodiment, the present invention can provide a method of determining a diagnosis of a subject in respect of a disease, the method comprising the steps of providing a SNP score for the subject as described herein; and correlating said SNP score to said subject diagnosis by determining if said SNP score is associated with a predisposition to said disease.

In still a further embodiment, the present invention can provide a method of determining whether or not a subject should undergo treatment for a disease, the method comprising the steps of providing a SNP score for the subject as described herein; and correlating said SNP score to said subject diagnosis by determining if said SNP score is associated with a predisposition to said disease.

In one embodiment, the determination of association of said SNP score with a predisposition to said disease is by reference to a distribution of SNP scores, preferably a distribution of SNP scores for disease sufferers, more preferably a distribution of SNP scores for both disease sufferers and non-sufferers.

Preferably, the treatment can be a diagnostic treatment, a therapeutic treatment, or a preventative treatment for the disease.

In yet a further embodiment, the present invention provides a method of assessing a subject's risk of developing two or more diseases, the method comprising the steps of

providing a net score for the subject as described herein in respect of each of the two or more diseases; and

combining the two or more net scores to give a combined score, said combined score representing the balance between the combined value of the subject's protective polymorphisms and the combined value of the subject's susceptibility polymorphisms for each of the two or more diseases;

wherein a combined protective score can be predictive of a reduced risk of developing the two or more diseases and a combined susceptibility score is predictive of an increased risk of developing the two or more diseases.

Preferably, the two or more diseases can be selected from the group comprising COPD, OCOPD, lung cancer, or ACS, more preferably the two or more diseases can be COPD, lung cancer and ACS.

In still a further aspect the present invention provides for the use of a combined score in the assessment of a subject's risk of developing two or more diseases, wherein the combined score represents the balance between the combined value of the subject's protective polymorphisms and the combined value of the subject's susceptibility polymorphisms for each of the two or more diseases;

and wherein a combined protective score is predictive of a reduced risk of developing the two or more diseases and a combined susceptibility score is predictive of an increased risk of developing the two or more diseases.

In further aspects the invention provides methods and uses substantially as herein described with or without reference to the examples.

As used herein “genetic analysis” means not only analysis directly at the nucleic acid level but also at the genetic-related analysis which can involve analysis of the level of expression and/or activity of a gene product, including on a proteomic basis.

Preferably, said disease or condition is selected from acquired diseases and conditions. “Acquired” diseases or conditions can be those which develop, or to which a predisposition is developed, primarily due to lifestyle and occupational events. Diseases or conditions which result from smoking can be one example of an acquired disease or condition.

Preferably, said data from said at least one genetic analysis can be combined with data indicative of a predisposition on the part of said subject to one or more diseases or conditions based upon the family, occupational, environmental or lifestyle history of said subject.

Preferably, said at least one genetic analysis can be selected from amongst genetic tests which predict the predisposition of the subject to one or more diseases selected from cancer (including lung cancer), coronary artery disease (including ACS), COPD, emphysema and OCOPD.

Preferably, said tests can be selected from the Emphagene™-brand pulmonary test (as herein defined), Respirogene™-brand pulmonary test (as herein defined), Bronchogene™-brand lung cancer test (as herein defined), Cardiogene™-brand cardiovascular test (as herein defined) and Combogene™-brand diagnostic test (as herein defined).

This invention can also be said broadly to consist in the parts, elements and features referred to or indicated in the specification of the application, individually or collectively, and any or all combinations of any two or more of said parts, elements or features, and where specific integers can be mentioned herein which have known equivalents in the art to which this invention relates, such known equivalents can be deemed to be incorporated herein as if individually set forth.

Preferred forms of the present invention will now be described with reference to the examples and the accompanying figures (the content of which is here incorporated).

### BRIEF DESCRIPTION OF FIGURES

FIG. 1 depicts a graph showing the frequency of COPD plotted against SNP score derived from the 9 SNP panel as described in Example 1.

FIG. 2 depicts a graph showing the distribution of frequencies of control smokers and COPD subjects plotted against SNP score derived from the 9 SNP panel as described in Example 1.

FIG. 3 depicts a graph showing the likelihood of having COPD plotted against the SNP score derived from the 9 SNP panel as described in Example 1.

FIG. 4 depicts a graph showing the distribution of frequencies of control smokers and COPD subjects plotted against SNP score derived from the 17 SNP panel as described in Example 1.

FIG. 5 depicts a graph showing the frequency of COPD plotted against the SNP score derived from the 17 SNP panel as described in Example 1.

FIG. 6: depicts a graph showing the frequency of lung cancer plotted against the SNP score derived from the 5 SNP panel as described in Example 2.

FIG. 7: depicts a graph showing the log odds of having lung cancer plotted against the SNP score derived from the 5 SNP panel as described in Example 2.

FIG. 8 depicts a graph showing the frequency of lung cancer plotted against the SNP score derived from the 11 SNP panel as described in Example 2.

FIG. 9 depicts a graph showing the percentage of individuals with lung cancer plotted against SNP score derived from the 11 SNP panel as described in Example 2. 95% confidence intervals were calculated using Wilson's method.

FIG. 10 depicts a graph showing the log odds of having lung cancer plotted against SNP score derived from the 11 SNP panel as described in Example 2.

FIG. 11 depicts a receiver-operator curve analysis of sensitivity and specificity for the 11 SNP panel as described in Example 2.

FIG. 12 depicts a graph showing the distribution of frequencies of control smokers and lung cancer subjects plotted against SNP score derived from the 11 SNP panel as described in Example 2.

FIG. 13 depicts a graph showing the frequency of lung cancer plotted against the SNP score derived from a 16 SNP panel as described in Example 2.

FIG. 14 depicts a receiver-operator curve analysis of sensitivity and specificity for the 16 SNP panel as described in Example 2.

FIG. 15 depicts a graph showing the distribution of frequencies of control smokers and lung cancer subjects plotted against SNP score derived from the 16 SNP panel as described in Example 2.

FIG. 16 depicts a graph showing the log odds of having lung cancer plotted against the SNP score derived from the 9 SNP panel described in Example 2.

FIG. 17 depicts a receiver-operator curve analysis of sensitivity and specificity for the 9 SNP panel as described in Example 2.

FIG. 18 depicts a graph showing the distribution of frequencies of control smokers and lung cancer subjects plotted against SNP score derived from the 9 SNP panel as described in Example 2.

FIG. 19 depicts a graph showing the distribution of frequencies of control smokers and ACS subjects plotted against SNP score derived from the 11 SNP panel as described in Example 3.

FIG. 20 depicts a graph showing the frequency of ACS plotted against the SNP score derived from the 11 SNP panel as described in Example 3.

FIG. 21 depicts a graph showing the distribution of frequencies of control smokers and ACS subjects plotted against SNP score derived from the 15 SNP panel as described in Example 3.

FIG. 22 depicts a graph showing the frequency of ACS plotted against the SNP score derived from the 15 SNP panel as described in Example 3.

FIG. 23 depicts a graph showing a distribution of combined scores for SNP tests for lung cancer (referred to herein as the Bronchogene™-brand lung cancer test), acute coronary syndrome (referred to herein as the Cardiogene™-brand cardiovascular test) and COPD (referred to herein as the Emphagene™-brand pulmonary test) amongst smokers as described in Example 6.

### DESCRIPTION OF THE PREFERRED EMBODIMENTS

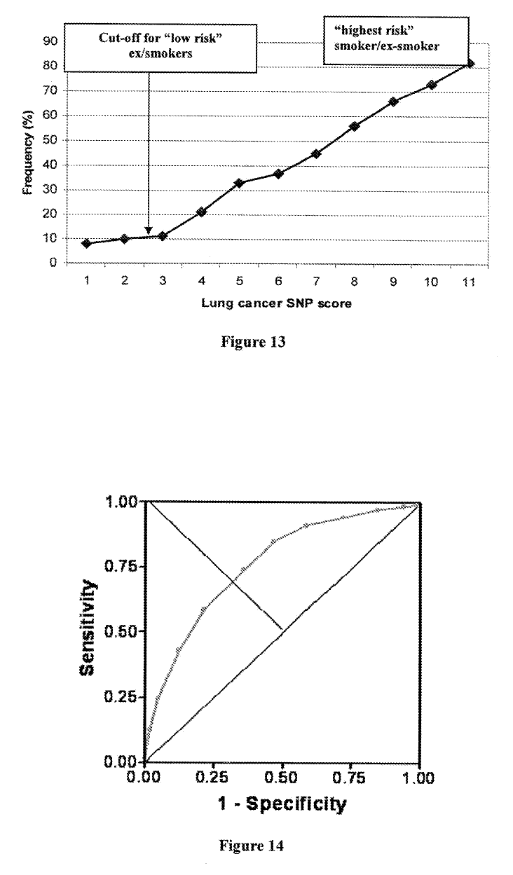

It is recognised that individual SNPs can confer weak risk of susceptibility or protection to a disease or phenotype of interest. These modest effects from individual SNPs can be typically measured as odds ratios in the order of 1-3. The specific phenotype of interest can be a disease, such as lung cancer, or an intermediate phenotype based on a pathological, biochemical or physiological abnormality (for example, impaired lung function). As shown herein, when specific genotypes from individual SNPs are assigned a numerical value reflecting their phenotypic effect (for example, a positive value for susceptibility SNPs and a negative value for protective SNPs), the combined effects of these SNPs can be derived from an algorithm that calculates an overall (or composite) score. Again as shown herein in a case-control study design, this SNP score is linearly related to the frequency of disease (or likelihood of having disease)—see, for example FIGS. 8 and 13. This is particularly evident when relevant environmental factors have been matched or adjusted for.

The risk can be based on frequency of disease or the odds ratio (OR) of disease risk. When the SNP score is plotted on the x axis and the risk of disease (OR or frequency of disease (%)) on the y axis, there is a linear relationship consistent with a dose effect—the higher the score the greater the risk. This is sharp contrast to the majority of genetic tests in clinical use today which are dichotomized as either positive or negative based on the presence or absence of specific genetic variants in a gene of interest. In this setting, these genetic tests can not be considered as yielding a continuous variable with low, medium and high values, and with such tests there is no linear relationship of risk based on the presence or absence of a specific genetic variant. Here, specific mutations can confer differing degrees of risk, particularly in different cohorts, but this is still based on merely the presence or absence of single mutations (genetic variants) in single genes.

In developing a genetic score based on a panel of polymorphisms from various genes that confers differing levels of risk, one has a tool more akin to a biochemical or physiological variable such as blood pressure or serum cholesterol level. These variables also exhibit a linear relationship with risk, so that the higher the value the greater the risk—for example, higher cholesterol or blood pressure is associated with greater risk of coronary heart disease or stroke. This type of variable has clinical utility in assigning a level of risk to individuals relative to others with different values—in the instant case relative to those with different genetic scores.

However, tests that define risk are not necessarily good at segmenting large groups of people into low risk groups and high risk groups with sufficient discrimination to allow subgroups of people to be prioritized for certain interventions such as screening, preventive lifestyle modification, preventive drug therapy or preventive surgery. A good example of this is shown by the poor utility of serum cholesterol in identifying which people are at risk of death from heart attack, as reported in Wald N J, et al., “When can a risk factor be used as a worthwhile screening test?” BMJ 319:1562-1565, (1999), which suggests that serum cholesterol is a poor discriminator of risk at a population level although it has utility for individuals. This is probably due to the fact that multiple factors confer risk of heart attack, and when one factor alone is used (like serum cholesterol) its effects across an entire pool of people are obscured by these other factors. Epidemiologists have attempted to improve this situation by developing analyses that consider many risk factors, such as the Framingham equations for heart disease which determine risk based on the combined effects of many parameters with each parameter conferring its own level of risk.

In complex diseases such as cancer, coronary artery disease, stroke, chronic obstructive lung disease, diabetes, obesity, arthritis or autoimmune diseases, there are believed to both genetic factors and environmental factors that are relevant to disease risk. Additionally, there is a belief that these diseases are genetically very heterogeneous. This means that specific polymorphisms within different genes can confer risk in different subgroups with the same disease. The SNP score described herein recognizes and allows for this by being based on a panel of SNPs, each contributing to the composite risk independently. In contrast to the approach exemplified by the Framingham equation, which is a composite score for several variables measurable in all people, the genetic SNP score is made up of polymorphisms in genes of highly variable frequency, so that rare SNPs that are found less often can be powerful discriminators of low and high risk. In contrast, common SNPs can confer less of a discriminatory power across populations.

In one embodiment, the SNP score can provide a means of comparing people with different scores and their odds of having disease in a simple dose-response relationship. In this analysis, the people with the lowest SNP score are the referent group (Odds ratio=1) and those with greater SNP scores have a correspondingly greater odds (or likelihood) of having the disease—again in a linear fashion. The Applicants believe, without wishing to be bound by any theory, that the extent to which combining SNPs optimises these analyses is dependent, at least in part, on the strength of the effect of each SNP individually in a univariate analysis (independent effect) and/or multivariate analysis (effect after adjustment for effects of other SNPs or non-genetic factors) and the frequency of the genotype from that SNP (how common the SNP is). However, the effect of combining certain SNPs can also be in part related to the effect that those SNPs have on certain pathophysiological pathways that underlie the phenotype or disease of interest.

When the utility of genetic SNP score in the segmentation of at risk populations into low, medium and high risk was assessed, it was surprisingly found that certain combinations of SNPs allowed more precise segmentation. The Applicants have found that combining certain SNPs can increase the accuracy of the determination of risk or likelihood of disease, and that the accuracy is not dependant solely on the number of SNPs comprising the panel. Specifically, when the distribution of SNP scores for the cases and controls are plotted according to their frequency, the ability to segment those with and without disease (or risk of disease) can be improved according to the specific combination of SNPs that are analysed. See, for example, the distributions of risk score for ACS as described in Example 3 herein, where a better segmentation of the population was observed with the 11 SNP panel (FIG. 19) compared to that observed with the 15 SNP panel (FIG. 21). It appears that this effect is not solely dependent on the number of relevant SNPs that are analysed in combination, nor the magnitude of their individual effects, nor their frequencies in the cases or controls. It further appears that the ability to improve this segmentation of the population into high and low risk is not due to any specific ratio of susceptibility or protective SNPs. The Applicants believe, without wishing to be bound by any theory, that the greater separation of the population in to high and low risk can at least partly be a function of identifying SNPs that confer a susceptibility or protective phenotype in important but independent pathophysiological pathways. The Applicants believe, without wishing to be bound by any theory, that certain SNPs have biologically independent effects that are preferably analysed in their entirety in order to have optimised segmentation. This would be consistent with the effects of genetic heterogeneity where many different effects, in different combination, are required before a disease develops.

When deriving a SNP score for each person, the score is the composite of any number of SNPs, with many SNPs making no contribution to the score—if the person does not carry the susceptibility or protective genetic variant for a specific SNP, the contribution of that SNP to the composite SNP score is 0. This is in sharp contrast to the multivariate analyses exemplified by the Framingham score.

Therefore, in addition to assigning risk to individuals based on their genetic SNP score, it is possible to segment a population when the frequency of the SNP score is compared between cases and controls and separation of the two distributions is achieved. The assignment of risk has utility in treating individuals (for example, prescribing a drug), whereas the segmentation of populations allows treatment strategies to be applied across populations (in for example a public health approach such as population-wide screening). Such treatment strategies can seek to optimise the application of one or more interventions amongst a population to achieve a given result, such as, for example, eradication of a communicable disease or to maximize cost-effectiveness. It should be noted that these separate utilities—the assignation of risk to an individual and the segmentation of a population—are independent of each other and the presence of the former does not predict the later (see, for example, Wald et al., (1999)).

This observation has therefore clinical utility in helping to define a threshold or cut-off level in the SNP score that will define a subgroup of the population who are candidates to undergo an intervention. Such an intervention can be a diagnostic intervention, such as imaging test, other screening or diagnostic test (for example, a biochemical or RNA based test), or can be a therapeutic intervention, such as a chemopreventive therapy (for example, cisplatin or etoposide for small cell lung cancer), radiotherapy, or a preventive lifestyle modification (stopping smoking for lung cancer). In defining this clinical threshold, people can be prioritised to a particular intervention in such a way to minimise costs or minimise risks of that intervention (for example, the costs of image-based screening or expensive preventive treatment or risk from drug side-effects or risk from radiation exposure). In determining this threshold, one might aim to maximise the ability of the test to detect the majority of cases (maximise sensitivity) but also to minimise the number of people at low risk that require, or can be are otherwise eligible for, the intervention of interest.

Receiver-operator curve (ROC) analyses analyze the clinical performance of a test by examining the relationship between sensitivity and false positive rate (i.e., 1-specificity) for a single variable in a given population. In an ROC analysis, the test variable can be derived from combining several factors. Either way, this type of analysis does not consider the frequency distribution of the test variable (for example, the SNP score) in the population and therefore the number of people who would need to be screened in order to identify the majority of those at risk but to minimise the number who need to be screened or treated. The Applicants have found that this frequency distribution plot appears to be dependent on the particular combination of SNPs under consideration and can not be predicted by the effect conferred by each SNP on its own nor from its performance characteristics (sensitivity and specificity) in an ROC analysis.

Based on observations across 3 different disease groups, the Applicants believe that this approach can be generalized to other diseases where there are multiple genetic factors (with or without environmental effects) that in different combinations independently confer disease or disease risk.

The data presented herein shows that determining a specific combination of SNPs can enhance the ability to segment or subgroup people into intervention and non-intervention groups in order to better prioritise these interventions. For example, such an approach is useful in identifying which smokers might be best prioritised for interventions, such as CT screening for lung cancer. Such an approach could also be used for initiating treatments or other screening or diagnostic tests. As will be appreciated, this has important cost implications to offering such interventions.

Accordingly, an embodiment of the present invention also provides a method of assessing a subject's suitability for an intervention diagnostic of or therapeutic for a disease, the method including:

a) providing a net score for said subject, wherein the net score is or has been determined by:

i) providing the result of one or more genetic tests of a sample from the subject, and analysing the result for the presence or absence of protective polymorphisms and for the presence or absence of susceptibility polymorphisms, wherein said protective and susceptibility polymorphisms are associated with said disease,

ii) assigning a positive score for each protective polymorphism and a negative score for each susceptibility polymorphism or vice versa;

iii) calculating a net score for said subject by representing the balance between the combined value of the protective polymorphisms and the combined value of the susceptibility polymorphisms present in the subject sample;

b) providing a distribution of net scores for disease sufferers and non-sufferers wherein the net scores for disease sufferers and non-sufferers are or have been determined in the same manner as the net score determined for said subject; and

c) determining whether the net score for said subject lies within a threshold on said distribution separating individuals deemed suitable for said intervention from those for whom said intervention is deemed unsuitable;

wherein a net score within said threshold is indicative of the subject's suitability for the intervention, and wherein a net score outside the threshold is indicative of the subject's unsuitability for the intervention.

The value assigned to each protective polymorphism can be the same or can be different. The value assigned to each susceptibility polymorphism can be the same or can be different, with either each protective polymorphism having a negative value and each susceptibility polymorphism having a positive value, or vice versa.

The intervention can be a diagnostic test for the disease, such as a blood test or a CT scan for lung cancer. Alternatively, the intervention can be a therapy for the disease, such as chemotherapy or radiotherapy, including a preventative therapy for the disease, such as the provision of motivation to the subject to stop smoking.

As described herein, a distribution of SNP scores for, for example, lung cancer sufferers and resistant smoker controls (non-sufferers) can be established using the methods of the invention. For example, a distribution of SNP scores derived from the 16 SNP panel consisting of the protective and susceptibility polymorphisms selected from the group consisting of the −133 G/C polymorphism in the Interleukin-18 gene, the −1053 C/T polymorphism in the CYP 2E1 gene, the Arg197gln polymorphism in the Nat2 gene, the −511 G/A polymorphism in the Interleukin 1B gene, the Ala 9 Thr polymorphism in the Anti-chymotrypsin gene, the S allele polymorphism in the Alpha1-antitrypsin gene, the −251 A/T polymorphism in the Interleukin-8 gene, the Lys 751 gln polymorphism in the XPD gene, the +760 G/C polymorphism in the SOD3 gene, the Phe257Ser polymorphism in the REV gene, the Z allele polymorphism in the Alpha1-antitrypsin gene, the R19W A/G polymorphism in the Cerberus 1 (Cer 1) gene, the Ser307Ser G/T polymorphism in the XRCC4 gene, the K3326X A/T polymorphism in the BRCA2 gene, the V433M A/G polymorphism in the Integrin alpha-11 gene, and the E375G T/C polymorphism in the CAMKK1 gene, among lung cancer sufferers and non-sufferers is described herein. As shown herein, a threshold SNP score can be determined that separates people into intervention and non-intervention groups, so as to better prioritise those individuals suitable for such interventions.

The predictive methods of the invention allow a number of therapeutic interventions and/or treatment regimens to be assessed for suitability and implemented for a given subject. The simplest of these can be the provision to the subject of motivation to implement a lifestyle change, for example, where the subject is a current smoker, the methods of the invention can provide motivation to quit smoking.

The manner of therapeutic intervention or treatment will be predicated by the nature of the polymorphism(s) and the biological effect of said polymorphism(s). For example, where a susceptibility polymorphism is associated with a change in the expression of a gene, intervention or treatment is preferably directed to the restoration of normal expression of said gene, by, for example, administration of an agent capable of modulating the expression of said gene. Where a polymorphism is associated with decreased expression of a gene, therapy can involve administration of an agent capable of increasing the expression of said gene, and conversely, where a polymorphism is associated with increased expression of a gene, therapy can involve administration of an agent capable of decreasing the expression of said gene. Methods useful for the modulation of gene expression are well known in the art. For example, in situations where a polymorphism is associated with upregulated expression of a gene, therapy utilising, for example, RNAi or antisense methodologies can be implemented to decrease the abundance of mRNA and so decrease the expression of said gene. Alternatively, therapy can involve methods directed to, for example, modulating the activity of the product of said gene, thereby compensating for the abnormal expression of said gene.

Where a susceptibility polymorphism is associated with decreased gene product function or decreased levels of expression of a gene product, therapeutic intervention or treatment can involve augmenting or replacing of said function, or supplementing the amount of gene product within the subject for example, by administration of said gene product or a functional analogue thereof. For example, where a polymorphism is associated with decreased enzyme function, therapy can involve administration of active enzyme or an enzyme analogue to the subject. Similarly, where a polymorphism is associated with increased gene product function, therapeutic intervention or treatment can involve reduction of said function, for example, by administration of an inhibitor of said gene product or an agent capable of decreasing the level of said gene product in the subject. For example, where a SNP allele or genotype is associated with increased enzyme function, therapy can involve administration of an enzyme inhibitor to the subject.

Likewise, when a protective polymorphism is associated with upregulation of a particular gene or expression of an enzyme or other protein, therapies can be directed to mimic such upregulation or expression in an individual lacking the resistive genotype, and/or delivery of such enzyme or other protein to such individual Further, when a protective polymorphism is associated with down-regulation of a particular gene, or with diminished or eliminated expression of an enzyme or other protein, desirable therapies can be directed to mimicking such conditions in an individual that lacks the protective genotype.

Embodiments of the present invention are directed to methods for the assessment of the suitability of a particular subject for an intervention, including diagnostic, therapeutic and preventative interventions, with respect to a particular disease. The methods rely upon the recognition that for many (if not all) diseases there exist genetic polymorphisms which fall into two categories—namely those indicative of a reduced risk of developing a particular disease (which can be termed “protective polymorphisms” or “protective SNPs”) and those indicative of an increased risk of developing a particular disease (which can be termed “susceptibility polymorphisms” or “susceptibility SNPs”).

As used herein, the phrase “assessing a subject's suitability for an intervention” or grammatical equivalents thereof means one or more determinations of whether a given subject is or should be a candidate for an intervention or is not or should not be a candidate for an intervention. Preferably, the assessment involves a determination of the subject's SNP score in relation to a distribution of SNP scores as described herein.

As used herein the term “intervention” includes medical tests, analyses, and treatments, including diagnostic, therapeutic and preventative treatments, and psychological or psychiatric tests, analyses and treatments, including counseling and the like.

As used herein, the phrase “risk of developing [a] disease” means the likelihood that a subject to whom the risk applies will develop the disease, and includes predisposition to, and potential onset of the disease. Accordingly, the phrase “increased risk of developing [a] disease” means that a subject having such an increased risk possesses an hereditary inclination or tendency to develop the disease. This does not mean that such a person will actually develop the disease at any time, merely that he or she has a greater likelihood of developing the disease compared to the general population of individuals that either does not possess a polymorphism associated with increased disease risk, or does possess a polymorphism associated with decreased disease risk. Subjects with an increased risk of developing the disease include those with a predisposition to the disease, for example in the case of COPD, a tendency or predilection regardless of their lung function at the time of assessment, for example, a subject who is genetically inclined to COPD but who has normal lung function, those at potential risk, for example in the case of COPD, subjects with a tendency to mildly reduced lung function who are likely to go on to suffer COPD if they keep smoking, and subjects with potential onset of the disease, for example in the case of COPD, subjects who have a tendency to poor lung function on spirometry etc., consistent with COPD at the time of assessment.

Similarly, the phrase “decreased risk of developing [a] disease” means that a subject having such a decreased risk possesses an hereditary disinclination or reduced tendency to develop the disease. This does not mean that such a person will not develop the disease at any time, merely that he or she has a decreased likelihood of developing the disease compared to the general population of individuals that either does possess one or more polymorphisms associated with increased disease risk, or does not possess a polymorphism associated with decreased disease risk.

It will be understood that in the context of the present invention the term “polymorphism” means the occurrence together in the same population at a rate greater than that attributable to random mutation (usually greater than 1%) of two or more alternate forms (such as alleles or genetic markers) of a chromosomal locus that differ in nucleotide sequence or have variable numbers of repeated nucleotide units. See www.ornl.gov/sci/techresources/Human_Genome/publicat/97pr/09gloss.html#p. Accordingly, the term “polymorphisms” is used herein contemplates genetic variations, including single nucleotide substitutions, insertions and deletions of nucleotides, repetitive sequences (such as microsatellites), and the total or partial absence of genes (e.g. null mutations). As used herein, the term “polymorphisms” also includes genotypes and haplotypes. A genotype is the genetic composition at a specific locus or set of loci. A haplotype is a set of closely linked genetic markers present on one chromosome which are not easily separable by recombination, tend to be inherited together, and can be in linkage disequilibrium. A haplotype can be identified by patterns of polymorphisms such as SNPs. Similarly, the term “single nucleotide polymorphism” or “SNP” in the context of the present invention includes single base nucleotide substitutions and short deletion and insertion polymorphisms. It will further be understood that the term “disease” is used herein in its widest possible sense, and includes conditions which can be considered disorders and/or illnesses which have a genetic basis or to which the genetic makeup of the subject contributes.

The phrase “determining the diagnosis” as used herein refers to methods by which the skilled artisan can predict the development of a condition in a patient. The term “diagnosis” does not refer to the ability to predict the development of a condition with 100% accuracy, or even that the development of the condition is more likely to occur than not. Instead, the skilled artisan will understand that the term “diagnosis” refers to an increased probability that a certain course or outcome (for example, onset of disease) will occur; that is, that a course or outcome is more likely to occur in a patient exhibiting a given characteristic, such as the presence or level of a diagnostic indicator, when compared to those individuals not exhibiting the characteristic. For example, as described hereinafter, a subject exhibiting a lung cancer SNP score greater than, for example, 8 can be more likely to develop lung cancer than a subject exhibiting a lower lung cancer SNP score. In a generalised example, in individuals not exhibiting the condition, the chance of developing the condition can be 3%. In such a case, the increased probability that the course or outcome will occur would be any number greater than 3%. In preferred embodiments, a diagnosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−1%.

A diagnosis is often determined by examining one or more “diagnostic indicators.” These are markers, the presence or amount of which in a patient (or a sample obtained from the patient) signal a probability that a given course or outcome (such as the development of a condition or disease) will occur. Diagnostic indicators associated with various diseases are well known in the art and are discussed further herein. For example, preferred diagnostic indicators in the diagnosis of diseases are SNP scores. For example, preferred diagnostic indicators in the diagnosis of ACS are the ACS SNP scores as described herein. When the SNP score (as calculated by the methods exemplified herein) reaches a sufficiently high level, the SNP score signals that the subject is at an increased risk of developing ACS, in comparison to a similar subject exhibiting a lower SNP score. A level of a diagnostic indicator, such as SNP scores, that signals an increased risk of disease is referred to as being “associated with an increased risk of disease” in a subject.

The skilled artisan will understand that associating a diagnostic indicator with a predisposition to a disease is a statistical analysis and can be determined by a level of statistical significance. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests for associating a diagnostic indicator with a predisposition to an adverse outcome are described herein.

The term “correlating” as used herein in reference to the use of diagnostic indicators to determine a diagnosis refers to comparing the presence or level of the diagnostic indicator in a patient to its presence or level in persons known to suffer from, or known to be at risk of, a given condition; or in persons known to be free of a given condition. For example, the age of a subject can be compared to ages known to be associated with an increased disposition to an age-related disease. The subject's age is said to have been correlated with a diagnosis; that is, the skilled artisan can use the subject's age to determine the likelihood that the patient is at risk for an age-related disease, and respond accordingly. Alternatively, the subject's age can be compared to ages known to be associated with a good outcome (e.g., decreased incidence of the age-related disease), thereby to determine a predisposition to the good outcome.

In certain embodiments, a diagnostic indicator is correlated to a subject diagnosis by merely its presence or absence. In other embodiments, a threshold level of a diagnostic indicator can be established, and the level of the indicator for a subject can simply be compared to the threshold level. For example, a SNP score for a subject can be established as a level at which a subject is at an increased disposition to a disease. For example, as described herein in Example 2, a preferred threshold level for SNP score on the 16 SNP lung cancer panel of the invention is about 4.

Using case-control studies, the frequencies of several genetic variants (polymorphisms) of candidate genes have been compared in disease sufferers, for example, in chronic obstructive pulmonary disease (COPD) sufferers, in occupational chronic obstructive pulmonary disease (OCOPD) sufferers, in lung cancer sufferers, in ACS sufferers, and in control subjects not suffering from the relevant disease, for example smokers without lung cancer and with normal lung function. The majority of these candidate genes have confirmed (or likely) functional effects on gene expression or protein function.

In various specific embodiments, the frequencies of polymorphisms between blood donor controls, resistant subjects and those with COPD, the frequencies of polymorphisms between blood donor controls, resistant subjects and those with OCOPD, the frequencies of polymorphisms between blood donor controls, resistant subjects and those with lung cancer, and the frequencies of polymorphisms between blood donor controls, resistant subjects and those with ACS have been compared. This has resulted in both protective and susceptibility polymorphisms being identified for each disease.

The surprising finding by the Applicant relevant to this invention is that a combined analysis of protective and susceptibility polymorphisms discriminatory for a given disease yields a result that is indicative of that subject's suitability for an intervention in relation to that disease. This approach is widely applicable, on a disease-by-disease basis.

The present invention identifies methods of assessing the suitability of a subject for an intervention in respect of a disease which comprises determining in said subject the presence or absence of protective and susceptibility polymorphisms associated with said disease. A net score for said subject is derived, said score representing the balance between the combined value of the protective polymorphisms present in said subject and the combined value of the susceptibility polymorphisms present in said subject. A net protective score is predictive of a reduced risk of developing said disease, and a net susceptibility score is predictive of an increased risk of developing said disease. Moreover, the net score can be used to establish the suitability of the subject for an intervention, by comparison with distributions of net scores for disease sufferers and non-sufferers.

Within each category (protective polymorphisms, susceptibility polymorphisms, respectively) the polymorphisms can each be assigned the same value. For example, in the analyses presented in the Examples herein, each protective polymorphism associated with a given disease is assigned a value of +1, and each susceptibility polymorphism is assigned a value of −1. Alternatively, polymorphisms discriminatory for a disease within the same category can each be assigned a different value to reflect their discriminatory value for said disease. For example, a polymorphism highly discriminatory of risk of developing a disease can be assigned a high weighting, for example a polymorphism with a high Odd's ratio can be considered highly discriminatory of disease, and can be assigned a high weighting.

The subject sample can have already been analysed for the presence or absence of one or more protective or susceptibility polymorphisms, and the determination of a net score comprises the steps of

assigning a positive score for each protective polymorphism and a negative score for each susceptibility polymorphism or vice versa;

calculating a net score for said subject, said net score representing the balance between the combined value of the protective polymorphisms and the combined value of the susceptibility polymorphisms present in the subject sample;

wherein a net protective score is predictive of a reduced risk of developing said disease and a net susceptibility score is predictive of an increased risk of developing said disease.

In one embodiment the at least one genetic analysis is the Emphagene™-brand pulmonary test. As used herein, the Emphagene™-brand pulmonary test comprises the methods of determining a subject's predisposition to and/or potential risk of developing chronic obstructive pulmonary disease (COPD) and/or emphysema and related methods as defined in New Zealand Patent Applications No. 539934, No. 541935, No. 545283, and PCT International Application PCT/NZ2006/000103 (published as WO2006/121351) each incorporated herein in its entirety.

In particular, the Emphagene™-brand pulmonary test includes a method of determining a subject's risk of developing one or more obstructive lung diseases comprising analysing a sample from said subject for the presence or absence of one or more polymorphisms selected from the group including:
−765 C/G in the promoter of the gene encoding Cyclooxygenase 2 (COX2);
105 C/A in the gene encoding Interleukin18 (IL18);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tissue Necrosis Factor α (TNFα);
C89Y A/G in the gene encoding SMAD3;
E 469 K A/G in the gene encoding Intracellular Adhesion molecule 1 (ICAM1);
Gly 881Arg G/C in the gene encoding Caspase (NOD2);
161 G/A in the gene encoding Mannose binding lectin 2 (MBL2);
−1903 G/A in the gene encoding Chymase 1 (CMA1);
Arg 197 Gln G/A in the gene encoding N-Acetyl transferase 2 (NAT2);
−366 G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1+49 C/T in the gene encoding Elafin; or
−1607 1G/2G in the promoter of the gene encoding Matrix Metalloproteinase 1 (MMP1),

with reference to the 1G allele only;

wherein the presence or absence of one or more of said polymorphisms can be indicative of the subject's risk of developing one or more obstructive lung diseases selected from the group consisting of chronic obstructive pulmonary disease (COPD), emphysema, or both COPD and emphysema.

The one or more polymorphisms can be detected directly or by detection of one or more polymorphisms which are in linkage disequilibrium with said one or more polymorphisms.

Linkage disequilibrium (LD) is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204).

Briefly, 17 susceptibility genetic polymorphisms and 19 protective genetic polymorphisms identified as discriminatory for COPD or emphysema were analysed using methods of the invention. These analyses can be used to determine the suitability of any subject for an intervention in respect of COPD or emphysema, and to identify those genetic polymorphisms of most use in determining a subject's risk of developing COPD or emphysema.

As used herein, the Bronchogene™-brand lung cancer test comprises the methods of determining a subject's predisposition to and/or potential risk of developing lung cancer and related methods as defined in New Zealand Patent Application Nos 540203, No. 541787, No. 543297, No. 550643, No. 554707, and PCT International Application PCT/NZ2006/000125 (published as WO2006/123955) each incorporated herein in their entirety.

In particular, the Bronchogene™-brand lung cancer test includes a method of determining a subject's risk of developing lung cancer comprising analysing a sample from said subject for the presence or absence of one or more polymorphisms selected from the group including:
Asp 298 Glu in the gene encoding Nitric oxide synthase 3 (NOS3);
−786 T/C in the promoter of the gene encoding NOS3;
Arg 312 Gln in the gene encoding Superoxide dismutase 3 (SOD3);
Ala 15 Thr in the gene encoding Anti-chymotrypsin (ACT);
Asn 357 Ser A/G in the gene encoding Matrix metalloproteinase 12 (MMP12);
105 A/C in the gene encoding Interleukin-18 (IL-18);
−133 G/C in the promoter of the gene encoding Interleukin-18;
874 A/T in the gene encoding Interferon γ (IFNγ);
−765 G/C in the gene encoding Cyclooxygenase 2 (COX2);
−447 G/C in the gene encoding Connective tissue growth factor (CTGF);
−221 C/T in the gene encoding Mucin 5AC (MUC5AC);
+161 G/A in the gene encoding Mannose binding lectin 2 (MBL2);
intron 1 C/T in the gene encoding Arginase 1 (Arg1);
Leu 252 Val C/G in the gene encoding Insulin-like growth factor II receptor (IGF2R); or
−1082 A/G in the gene encoding Interleukin 10 (IL-10);
A/T c74delA in the gene encoding cytochrome P450 polypeptide CYP3A43 (CYP3A43);
A/C (rs2279115) in the gene encoding B-cell CLL/lymphoma 2 (BCL2);
A/G at +3100 in the 3′UTR (rs2317676) of the gene encoding Integrin beta 3 (ITGB3);
−3714 G/T (rs6413429) in the gene encoding Dopamine transporter 1 (DAT1);
A/G (rs1139417) in the gene encoding Tumor necrosis factor receptor 1 (TNFR1);
C/Del (rs1799732) in the gene encoding Dopamine receptor D2 (DRD2);
C/T (rs763110) in the gene encoding Fas ligand (FasL);
C/T (rs5743836) in the gene encoding Toll-like receptor 9 (TLR9); or
−81 C/T (rs2273953) in the 5′ UTR of the gene encoding Tumor protein P73 (TP73);

wherein the presence or absence of one or more of said polymorphisms can be indicative of the subject's risk of developing lung cancer.

Again, the one or more polymorphisms can be detected directly or by detection of one or more polymorphisms which are in linkage disequilibrium with said one or more polymorphisms.

Briefly, 19 susceptibility genetic polymorphisms and 17 protective genetic polymorphisms, and subsequently 8 additional susceptibility genetic polymorphisms and 6 additional protective genetic polymorphism identified as discriminatory for lung cancer were analysed using methods of the invention. These analyses can be used to determine the suitability of any subject for an intervention in respect of lung cancer, and to identify those genetic polymorphisms of most use in determining a subject's risk of developing lung cancer.

As used herein, the Respirogene™-brand pulmonary test comprises the methods of determining a subject's predisposition to and/or potential risk of developing occupational chronic obstructive pulmonary disease (OCOPD) and related methods as defined in New Zealand Patent Applications No. 540202, No. 541389, and PCT International Application PCT/NZ2006/000124 (published as WO2006/123954) each incorporated herein in their entirety.

In particular, the Respirogene™-brand pulmonary test includes a method of determining a subject's risk of developing occupational chronic obstructive pulmonary disease comprising analysing a sample from said subject for the presence or absence of one or more polymorphisms selected from the group including:
−765 C/G in the promoter of the gene encoding cyclooxygenase 2 (COX2);
Ile 105 Val (A/G) in the gene encoding glutathione S transferase P (GSTP1);
105 C/A in the gene encoding interleukin-18 (IL-18);
−133 G/C in the promoter of the gene encoding IL-18;
−251 A/T in the gene encoding interleukin-8 (IL-8);
Lys 420 Thr (A/C) in the gene encoding Vitamin D binding protein (VDBP);
Glu 416 Asp (T/G) in the gene encoding VDBP;
exon 3 T/C (R/r) in the gene encoding microsomal epoxide hydrolase (MEH);
Arg 312 Gln (AC) in the gene encoding superoxide dismutase 3 (SOD3);
3′ 1237 G/A (T/t) in the gene encoding α1-antitrypsin;
α1-antitrypsin (α1AT) S polymorphism;
Asp 299 Gly A/G in the gene encoding toll-like receptor 4 (TLR4);
Gln27Glu in the gene encoding β2 adrenoreceptor (ADRB2);
−518 G/A in the promoter of the gene encoding interleukin-11 (IL-11);
−1055 (C/T) in the promoter of the gene encoding interleukin-13 (IL-13);
−675 4G/5G in the promoter of the gene encoding plasminogen activator inhibitor 1 (PAI-1);
298 Asp/Glu (T/G) in the gene encoding nitric oxide synthase 3 (NOS3);
−1607 1G/2G in the gene encoding matrix metalloproteinase 1 (MMP1);

wherein the presence or absence of one or more of said polymorphisms can be indicative of the subject's risk of developing occupational chronic obstructive pulmonary disease.

Again, the one or more polymorphisms can be detected directly or by detection of one or more polymorphisms which are in linkage disequilibrium with said one or more polymorphisms.

As used herein, the Cardiogene™-brand cardiovascular test comprises the methods of determining a subject's predisposition to and/or potential risk of developing acute coronary syndrome (ACS) and related methods as defined in New Zealand Patent Application No. 543520, No. 543985, No. 549951, and PCT International Application PCT/NZ2006/000292 each incorporated herein in their entirety.

In particular, the Cardiogene™-brand cardiovascular test includes a method of determining a subject's risk of developing ACS comprising analysing a sample from said subject for the presence or absence of one or more polymorphisms selected from the group consisting of:
−1903 A/G in the gene encoding Chymase 1 (CMA1);
−82 A/G in the gene encoding Matrix metalloproteinase 12 (MMP12);
Ser52Ser (223 C/T) in the gene encoding Fibroblast growth factor 2 (FGF2);
Q576R A/G in the gene encoding Interleukin 4 receptor alpha (IL4RA);
HOM T2437C in the gene encoding Heat Shock Protein 70 (HSP 70);
874 A/T in the gene encoding Interferon γ (IFNG);
−589 C/T in the gene encoding Interleukin 4 (IL-4);
−1084 A/G (−1082) in the gene encoding Interleukin 10 (IL-10);
Arg213Gly C/G in the gene encoding Superoxide dismutase 3 (SOD3);
459 C/T Intron I in the gene encoding Macrophage inflammatory protein 1 alpha (MIP1A);
Asn 125 Ser A/G in the gene encoding Cathepsin G;
I249V C/T in the gene encoding Chemokine (CX3C motif) receptor 1 (CX3CR1);
Gly 881 Arg G/C in the gene encoding Caspase (NOD2); or
372 T/C in the gene encoding Tissue inhibitor of metalloproteinase 1 (TIMP1);

wherein the presence or absence of one or more of said polymorphisms can be indicative of the subject's risk of developing ACS.

The one or more polymorphisms can be detected directly or by detection of one or more polymorphisms which are in linkage disequilibrium with said one or more polymorphisms.

As used herein, the Combogene™-brand diagnostic test comprises the methods of assessing the susceptibility of a subject to a disease and related methods as defined in New Zealand Patent Applications No. 540249, No. 541842, No. 551534, and PCT International Application PCT/NZ2006/000104 (published as WO2006/123943) each incorporated herein in their entirety.

In particular, the Combogene™-brand diagnostic test includes a method of assessing a subject's risk of developing a disease which includes:

analyzing a biological sample from said subject for the presence or absence of protective polymorphisms and for the presence or absence of susceptibility polymorphisms, wherein said protective and susceptibility polymorphisms are associated with said disease;

assigning a positive score for each protective polymorphism and a negative score for each susceptibility polymorphism or vice versa;

calculating a net score for said subject, said net score representing the balance between the combined value of the protective polymorphisms and the combined value of the susceptibility polymorphisms present in the subject sample;

wherein a net protective score can be predictive of a reduced risk of developing said disease and a net susceptibility score is predictive of an increased risk of developing said disease.

The value assigned to each protective polymorphism can be the same or can be different. The value assigned to each susceptibility polymorphism can be the same or can be different, with either each protective polymorphism having a negative value and each susceptibility polymorphism having a positive value, or vice versa.

Furthermore, the Combogene™-brand diagnostic test includes a method of determining a subject's risk of developing a disease, said method including

obtaining the result of one or more analyses of a sample from said subject to determine the presence or absence of protective polymorphisms and the presence or absence of susceptibility polymorphisms, and wherein said protective and susceptibility polymorphisms are associated with said disease;

assigning a positive score for each protective polymorphism and a negative score for each susceptibility polymorphism or vice versa;

calculating a net score for said subject, said net score representing the balance between the combined value of the protective polymorphisms and the combined value of the susceptibility polymorphisms present in the subject sample;

wherein a net protective score can be predictive of a reduced risk of developing said disease and a net susceptibility score is predictive of an increased risk of developing said disease.

In the case of each of the Emphagene™-brand pulmonary test, Bronchogene™-brand lung cancer test, Respirogene™-brand pulmonary test, Cardiogene™-brand cardiovascular test and Combogene™-brand diagnostic test, the “result” will normally be a categorisation of the genetic test outcome as indicative of the subject having a predisposition to the disease or condition which is greater than average (an increased predisposition), average (a neutral predisposition) or less than average (a reduced predisposition). Commonly, the categorisation will be made following a comparison of the raw data with a reference genetic database made up of data from a statistically-relevant number of similar tests performed previously and for which the association between specific genetic sequences and the presence or absence of disease is known. In preferred embodiments, the database will include specific polymorphic information, with individual polymorphisms being associated with either an increased predisposition to a disease or to a reduced predisposition to a disease. In alternative embodiments, the categorisation will be a determination of whether a net score for the subject lies within a threshold on a distribution of net scores determined for disease sufferers and non-sufferers, said threshold separating individuals having an increased predisposition from those individuals having a decreased predisposition.

In a further embodiment described herein in Example 3, susceptibility genetic polymorphisms and protective genetic polymorphisms identified as discriminatory for acute coronary syndrome (ACS) are analysed using methods of the invention. These analyses can be used to determine the risk quotient of any subject for ACS, and in particular to identify subjects at greater risk of developing ACS. The disorders herein collectively referred to as ACS are coronary or vascular disorders believed to be associated with inflammation, plaque instability, and/or smoking. ACS includes myocardial infarction and unstable angina.

Susceptibility and protective polymorphisms can readily be identified for other diseases using approaches similar to those described in the Examples, as well as in PCT International Application No. PCT/NZ02/00106 (published as WO 02/099134 and incorporated by reference) via which four susceptibility and three protective polymorphisms discriminatory for lung disease were identified.

The one or more polymorphisms can be detected directly or by detection of one or more polymorphisms which are in linkage disequilibrium with said one or more polymorphisms. As discussed above, linkage disequilibrium is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204).

Examples of polymorphisms reported to be in linkage disequilibrium are presented herein, and include the Interleukin-18-133 C/G and 105 A/C polymorphisms, and the Vitamin D binding protein Glu 416 Asp and Lys 420 Thr polymorphisms, as shown below.

| | | | | LD | |
|---|---|---|---|---|---|
| | | rs | Alleles | between | Phenotype in |
| Gene | SNP | numbers | in LD | alleles | COPD |
| IL-18 | −133 C/G | rs360721 | C allele | Strong LD | CC susceptible |
| | 105 A/C | rs549908 | A allele | | AA susceptible |
| VDBP | Lys 420 Thr | rs4588 | A allele | Strong LD | AA/AC |
| | | | | | protective |
| | Glu 416 Asp | rs7041 | T allele | | TT/TG |
| | | | | | protective |

It will be apparent that polymorphisms in linkage disequilibrium with one or more other polymorphism associated with increased or decreased risk of developing a disease, for example COPD, emphysema, or both COPD and emphysema will also provide utility as biomarkers for risk of developing the disease, for example COPD, emphysema, or both COPD and emphysema. The data presented herein shows that the frequency for SNPs in linkage disequilibrium is very similar. Accordingly, these genetically linked SNPs can be utilized in combined polymorphism analyses to derive a level of risk comparable to that calculated from the original SNP.

It will therefore be apparent that one or more polymorphisms in linkage disequilibrium with the polymorphisms specified herein can be identified, for example, using public data bases. Examples of such polymorphisms reported to be in linkage disequilibrium with the polymorphisms specified herein are presented in New Zealand Patent Applications No. 539934, No. 541935, No. 545283, PCT International Application PCT/NZ2006/000103 (published as WO2006/121351), New Zealand Patent Application Nos 540203, No. 541787, No. 543297, No. 550643, No. 554707, PCT International Application PCT/NZ2006/000125 (published as WO2006/123955), New Zealand Patent Applications No. 540202, No. 541389, PCT International Application PCT/NZ2006/000124 (published as WO2006/123954), New Zealand Patent Application No. 543520, No. 543985, No. 549951, PCT International Application PCT/NZ2006/000292, New Zealand Patent Applications No. 540249, No. 541842, No. 551534, and PCT International Application PCT/NZ2006/000104 (published as WO2006/123943).

The methods of the invention are primarily reliant on genetic information such as that derived from methods suitable to the detection and identification of single nucleotide polymorphisms (SNPs) associated with the specific disease for which a risk assessment is desired. SNP is a single base change or point mutation resulting in genetic variation between individuals. SNPs occur in the human genome approximately once every 100 to 300 bases, and can occur in coding or non-coding regions. Due to the redundancy of the genetic code, a SNP in the coding region can or can not change the amino acid sequence of a protein product. A SNP in a non-coding region can, for example, alter gene expression by, for example, modifying control regions such as promoters, transcription factor binding sites, processing sites, ribosomal binding sites, and affect gene transcription, processing, and translation.

SNPs can facilitate large-scale association genetics studies, and there has recently been great interest in SNP discovery and detection. SNPs show great promise as markers for a number of phenotypic traits (including latent traits), such as for example, disease propensity and severity, wellness propensity, and drug responsiveness including, for example, susceptibility to adverse drug reactions. Knowledge of the association of a particular SNP with a phenotypic trait, coupled with the knowledge of whether an individual has said particular SNP, can enable the targeting of diagnostic, preventative and therapeutic applications to allow better disease management, to enhance understanding of disease states and to ultimately facilitate the discovery of more effective treatments, such as personalised treatment regimens.

Indeed, a number of databases have been constructed of known SNPs, and for some such SNPs, the biological effect associated with a SNP. For example, the NCBI SNP database “dbSNP” is incorporated into NCBI's Entrez system and can be queried using the same approach as the other Entrez databases such as PubMed and GenBank. This database has records for over 1.5 million SNPs mapped onto the human genome sequence. Each dbSNP entry includes the sequence context of the polymorphism (i.e., the surrounding sequence), the occurrence frequency of the polymorphism (by population or individual), and the experimental method(s), protocols, and conditions used to assay the variation, and can include information associating a SNP with a particular phenotypic trait.

At least in part because of the potential impact on health and wellness, there has been and continues to be a great deal of effort to develop methods that reliably and rapidly identify SNPs. This is no trivial task, at least in part because of the complexity of human genomic DNA, with a haploid genome of 3×10⁹ base pairs, and the associated sensitivity and discriminatory requirements.

Genotyping approaches to detect SNPs well-known in the art include DNA sequencing, methods that require allele specific hybridization of primers or probes, allele specific incorporation of nucleotides to primers bound close to or adjacent to the polymorphisms (often referred to as “single base extension”, or “minisequencing”), allele-specific ligation (joining) of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation is also possible where the SNP lies in a coding region and results in an amino acid change.

DNA sequencing allows the direct determination and identification of SNPs. The benefits in specificity and accuracy are generally outweighed for screening purposes by the difficulties inherent in whole genome, or even targeted subgenome, sequencing.

Mini-sequencing involves allowing a primer to hybridize to the DNA sequence adjacent to the SNP site on the test sample under investigation. The primer is extended by one nucleotide using all four differentially tagged fluorescent dideoxynucleotides (A, C, G, or T), and a DNA polymerase. Only one of the four nucleotides (homozygous case) or two of the four nucleotides (heterozygous case) is incorporated. The base that is incorporated is complementary to the nucleotide at the SNP position.

A number of methods currently used for SNP detection involve site-specific and/or allele-specific hybridisation. These methods are largely reliant on the discriminatory techniques of Affymetrix (Santa Clara, Calif.) and Nanogen Inc. (San Diego, Calif.) are binding of oligonucleotides to target sequences containing the SNP of interest. The particularly well-known, and utilize the fact that DNA duplexes containing single base mismatches are much less stable than duplexes that are perfectly base-paired. The presence of a matched duplex is detected by fluorescence.

The majority of methods to detect or identify SNPs by site-specific hybridisation require target amplification by methods such as PCR to increase sensitivity and specificity (see, for example U.S. Pat. No. 5,679,524, PCT publication WO 98/59066, PCT publication WO 95/12607). US Application 20050059030 (incorporated herein in its entirety) describes a method for detecting a single nucleotide polymorphism in total human DNA without prior amplification or complexity reduction to selectively enrich for the target sequence, and without the aid of any enzymatic reaction. The method utilises a single-step hybridization involving two hybridization events: hybridization of a first portion of the target sequence to a capture probe, and hybridization of a second portion of said target sequence to a detection probe. Both hybridization events happen in the same reaction, and the order in which hybridisation occurs is not critical.

US Application 20050042608 (incorporated herein in its entirety) describes a modification of the method of electrochemical detection of nucleic acid hybridization of Thorp et al. (U.S. Pat. No. 5,871,918). Briefly, capture probes are designed, each of which has a different SNP base and a sequence of probe bases on each side of the SNP base. The probe bases are complementary to the corresponding target sequence adjacent to the SNP site. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. These differences in the oxidation rates at the different electrodes are used to determine whether the selected nucleic acid target has a single nucleotide polymorphism at the selected SNP site.

The technique of Lynx Therapeutics (Hayward, Calif.) using MEGATYPE™ technology can genotype very large numbers of SNPs simultaneously from small or large pools of genomic material. This technology uses fluorescently labeled probes and compares the collected genomes of two populations, enabling detection and recovery of DNA fragments spanning SNPs that distinguish the two populations, without requiring prior SNP mapping or knowledge.

A number of other methods for detecting and identifying SNPs exist. These include the use of mass spectrometry, for example, to measure probes that hybridize to the SNP. This technique varies in how rapidly it can be performed, from a few samples per day to a high throughput of 40,000 SNPs per day, using mass code tags. A preferred example is the use of mass spectrometric determination of a nucleic acid sequence which comprises the polymorphisms of the invention, for example, which includes the promoter of the COX2 gene or a complementary sequence. Such mass spectrometric methods are known to those skilled in the art, and the genotyping methods of the invention are amenable to adaptation for the mass spectrometric detection of the polymorphisms of the invention, for example, the COX2 promoter polymorphisms of the invention.

SNPs can also be determined by ligation-bit analysis. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′ end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174.

U.S. Pat. No. 6,821,733 (incorporated herein in its entirety) describes methods to detect differences in the sequence of two nucleic acid molecules that includes the steps of: contacting two nucleic acids under conditions that allow the formation of a four-way complex and branch migration; contacting the four-way complex with a tracer molecule and a detection molecule under conditions in which the detection molecule is capable of binding the tracer molecule or the four-way complex; and determining binding of the tracer molecule to the detection molecule before and after exposure to the four-way complex. Competition of the four-way complex with the tracer molecule for binding to the detection molecule indicates a difference between the two nucleic acids.

Protein- and proteomics-based approaches are also suitable for polymorphism detection and analysis. Polymorphisms which result in or are associated with variation in expressed proteins can be detected directly by analysing said proteins. This typically requires separation of the various proteins within a sample, by, for example, gel electrophoresis or HPLC, and identification of said proteins or peptides derived therefrom, for example by NMR or protein sequencing such as chemical sequencing or more prevalently mass spectrometry. Proteomic methodologies are well known in the art, and have great potential for automation. For example, integrated systems, such as the ProteomIQ™ system from Proteome Systems, provide high throughput platforms for proteome analysis combining sample preparation, protein separation, image acquisition and analysis, protein processing, mass spectrometry and bioinformatics technologies.

The majority of proteomic methods of protein identification utilise mass spectrometry, including ion trap mass spectrometry, liquid chromatography (LC) and LC/MSn mass spectrometry, gas chromatography (GC) mass spectroscopy, Fourier transform-ion cyclotron resonance-mass spectrometer (FT-MS), MALDI-TOF mass spectrometry, and ESI mass spectrometry, and their derivatives. Mass spectrometric methods are also useful in the determination of post-translational modification of proteins, such as phosphorylation or glycosylation, and thus have utility in determining polymorphisms that result in or are associated with variation in post-translational modifications of proteins.

Associated technologies are also well known, and include, for example, protein processing devices such as the “Chemical Inkjet Printer” comprising piezoelectric printing technology that allows in situ enzymatic or chemical digestion of protein samples electroblotted from 2-D PAGE gels to membranes by jetting the enzyme or chemical directly onto the selected protein spots. After in-situ digestion and incubation of the proteins, the membrane can be placed directly into the mass spectrometer for peptide analysis.

A large number of methods reliant on the conformational variability of nucleic acids have been developed to detect SNPs.

For example, Single Strand Conformational Polymorphism (SSCP, Orita et al., PNAS 1989 86:2766-2770) is a method reliant on the ability of single-stranded nucleic acids to form secondary structure in solution under certain conditions. The secondary structure depends on the base composition and can be altered by a single nucleotide substitution, causing differences in electrophoretic mobility under nondenaturing conditions. The various polymorphs are typically detected by autoradiography when radioactively labelled, by silver staining of bands, by hybridisation with detectably labelled probe fragments or the use of fluorescent PCR primers which are subsequently detected, for example by an automated DNA sequencer.

Modifications of SSCP are well known in the art, and include the use of differing gel running conditions, such as for example differing temperature, or the addition of additives, and different gel matrices. Other variations on SSCP are well known to the skilled artisan, including, RNA-SSCP, restriction endonuclease fingerprinting-SSCP, dideoxy fingerprinting (a hybrid between dideoxy sequencing and SSCP), bi-directional dideoxy fingerprinting (in which the dideoxy termination reaction is performed simultaneously with two opposing primers), and Fluorescent PCR-SSCP (in which PCR products are internally labelled with multiple fluorescent dyes, can be digested with restriction enzymes, followed by SSCP, and analysed on an automated DNA sequencer able to detect the fluorescent dyes).

Other methods which utilise the varying mobility of different nucleic acid structures include Denaturing Gradient Gel Electrophoresis (DGGE), Temperature Gradient Gel Electrophoresis (TGGE), and Heteroduplex Analysis (HET). Here, variation in the dissociation of double stranded DNA (for example, due to base-pair mismatches) results in a change in electrophoretic mobility. These mobility shifts are used to detect nucleotide variations.

Denaturing High Pressure Liquid Chromatography (HPLC) is yet a further method utilised to detect SNPs, using HPLC methods well-known in the art as an alternative to the separation methods described above (such as gel electrophoresis) to detect, for example, homoduplexes and heteroduplexes which elute from the HPLC column at different rates, thereby enabling detection of mismatch nucleotides and thus SNPs.

Yet further methods to detect SNPs rely on the differing susceptibility of single stranded and double stranded nucleic acids to cleavage by various agents, including chemical cleavage agents and nucleolytic enzymes. For example, cleavage of mismatches within RNA:DNA heteroduplexes by RNase A, of heteroduplexes by, for example bacteriophage T4 endonuclease YII or T7 endonuclease I, of the 5′ end of the hairpin loops at the junction between single stranded and double stranded DNA by cleavase I, and the modification of mispaired nucleotides within heteroduplexes by chemical agents commonly used in Maxam-Gilbert sequencing chemistry, are all well known in the art.

Further examples include the Protein Translation Test (PTT), used to resolve stop codons generated by variations which lead to a premature termination of translation and to protein products of reduced size, and the use of mismatch binding proteins. Variations are detected by binding of, for example, the MutS protein, a component of Escherichia coli DNA mismatch repair system, or the human hMSH2 and GTBP proteins, to double stranded DNA heteroduplexes containing mismatched bases. DNA duplexes are then incubated with the mismatch binding protein, and variations are detected by mobility shift assay. For example, a simple assay is based on the fact that the binding of the mismatch binding protein to the heteroduplex protects the heteroduplex from exonuclease degradation.

Those skilled in the art will know that a particular SNP, particularly when it occurs in a regulatory region of a gene such as a promoter, can be associated with altered expression of a gene. Altered expression of a gene can also result when the SNP is located in the coding region of a protein-encoding gene, for example where the SNP is associated with codons of varying usage and thus with tRNAs of differing abundance. Such altered expression can be determined by methods well known in the art, and can thereby be employed to detect such SNPs. Similarly, where a SNP occurs in the coding region of a gene and results in a non-synonymous amino acid substitution, such substitution can result in a change in the function of the gene product. Similarly, in cases where the gene product is an RNA, such SNPs can result in a change of function in the RNA gene product. Any such change in function, for example as assessed in an activity or functionality assay, can be employed to detect such SNPs.

The above methods of detecting and identifying SNPs are amenable to use in the methods of the invention.

In practicing the present invention to assess the risk a particular subject faces with respect to a particular disease, that subject will be assessed to determine the presence or absence of polymorphisms (preferably SNPs) which are either associated with protection from the disease or susceptibility to the disease.

In order to detect and identify SNPs in accordance with the invention, a sample containing material to be tested is obtained from the subject. The sample can be any sample potentially containing the target SNPs (or target polypeptides, as the case can be) and obtained from any bodily fluid (blood, urine, saliva, etc) biopsies or other tissue preparations.

DNA or RNA can be isolated from the sample according to any of a number of methods well known in the art. For example, methods of purification of nucleic acids are described in Tijssen; Laboratory Techniques in Biochemistry and Molecular Biology: Hybridization with nucleic acid probes Part 1: Theory and Nucleic acid preparation, Elsevier, New York, N.Y. 1993, as well as in Maniatis, T., Fritsch, E. F. and Sambrook, J., Molecular Cloning Manual 1989.

Upon detection of the presence or absence of the polymorphisms tested for, the critical step is to determine a net susceptibility score for the subject. This score will represent the balance between the combined value of the protective polymorphisms present and the total value of the susceptibility polymorphisms present, with a net protective score (i.e., a greater weight of protective polymorphisms present than susceptibility polymorphisms) being predictive of a reduced risk of developing the disease in question. The reverse is true where there is a net susceptibility score. To calculate where the balance lies, the individual polymorphisms are assigned a value. In the simplest embodiment, each polymorphisms within a category (i.e. protective or susceptibility) is assigned an equal value, with each protective polymorphism being −1 and each susceptibility polymorphism being +1 (or vice versa). It is however contemplated that the values assigned to individual polymorphisms within a category can differ, with some polymorphisms being assigned a value that reflects their predictive or discriminatory value. For example, one particularly strong protective polymorphism can have a value of −2, whereas another more weakly protective polymorphism can have a value of −0.75.

The net score, and the associated predictive outcome in terms of the risk of the subject developing a particular disease, can be represented in a number of ways. One example is as a graph as more particularly exemplified herein.

Another example is a simple numerical score (e.g. +2 to represent a subject with a net susceptibility score or −2 to represent a subject with a net protective score). In each case, the result is communicated to the subject with an explanation of what that result means to that subject. Preferably, advice on ways the subject can change their lifestyle so as to reduce the risk of developing the disease is also communicated to the subject.

It will be appreciated that the methods of the invention can be performed in conjunction with an analysis of other risk factors known to be associated with a disease, such as COPD, emphysema, OCOPD, lung cancer, or ACS. Such risk factors include epidemiological risk factors associated with an increased risk of developing the disease. Such risk factors include, but are not limited to smoking and/or exposure to tobacco smoke, age, sex and familial history. These risk factors can be used to augment an analysis of one or more polymorphisms as herein described when assessing a subject's risk of developing a disease such as COPD, emphysema, OCOPD, lung cancer or ACS. Examples of such combined analyses are described herein in the Examples.

The predictive methods of the invention allow a number of therapeutic interventions and/or treatment regimens to be assessed for suitability and implemented for a given subject, depending upon the disease and the overall risk quotient. The simplest of these can be the provision to a subject with a net susceptibility score of motivation to implement a lifestyle change, for example, in the case of OCOPD, to reduce exposure to aero-pollutants, for example, by an occupational change or by the use of safety equipment in the work place. Similarly where the subject is a current smoker, the methods of the invention can provide motivation to quit smoking. In this latter case, a ‘quit smoking’ program can be followed, which can include the use of anti-smoking medicaments (such as nicotine patches and the like) as well as anti-addiction medicaments.

Other therapeutic interventions can involve altering the balance between protective and susceptibility polymorphisms towards a protective state (such as by neutralizing or reversing a susceptibility polymorphism). The manner of therapeutic intervention or treatment will be predicated by the nature of the polymorphism(s) and the biological effect of said polymorphism(s). For example, where a susceptibility polymorphism is associated with a change in the expression of a gene, intervention or treatment is preferably directed to the restoration of normal expression of said gene, by, for example, administration of an agent capable of modulating the expression of said gene. Where a polymorphism, such as a SNP allele or genotype, is associated with decreased expression of a gene, therapy can involve administration of an agent capable of increasing the expression of said gene, and conversely, where a polymorphism is associated with increased expression of a gene, therapy can involve administration of an agent capable of decreasing the expression of said gene. Methods useful for the modulation of gene expression are well known in the art. For example, in situations were a polymorphism is associated with upregulated expression of a gene, therapy utilising, for example, RNAi or antisense methodologies can be implemented to decrease the abundance of mRNA and so decrease the expression of said gene. Alternatively, therapy can involve methods directed to, for example, modulating the activity of the product of said gene, thereby compensating for the abnormal expression of said gene.

Where a susceptibility polymorphism is associated with decreased gene product function or decreased levels of expression of a gene product, therapeutic intervention or treatment can involve augmenting or replacing of said function, or supplementing the amount of gene product within the subject for example, by administration of said gene product or a functional analogue thereof. For example, where a polymorphism is associated with decreased enzyme function, therapy can involve administration of active enzyme or an enzyme analogue to the subject. Similarly, where a polymorphism is associated with increased gene product function, therapeutic intervention or treatment can involve reduction of said function, for example, by administration of an inhibitor of said gene product or an agent capable of decreasing the level of said gene product in the subject. For example, where a polymorphism is associated with increased enzyme function, therapy can involve administration of an enzyme inhibitor to the subject.

Likewise, when a protective polymorphism is associated with upregulation of a particular gene or expression of an enzyme or other protein, therapies can be directed to mimic such upregulation or expression in an individual lacking the resistive genotype, and/or delivery of such enzyme or other protein to such individual Further, when a protective polymorphism is associated with down-regulation of a particular gene, or with diminished or eliminated expression of an enzyme or other protein, desirable therapies can be directed to mimicking such conditions in an individual that lacks the protective genotype.

The genetic analysis can provide results of two or more of the Emphagene™-brand pulmonary test, Respirogene™-brand pulmonary test, Bronchogene™-brand lung cancer test, Cardiogene™-brand cardiovascular test and Combogene™-brand diagnostic test. However, in other embodiments one or more of the Emphagene™-brand pulmonary test, Respirogene™-brand pulmonary test, Bronchogene™-brand lung cancer test, Cardiogene™-brand cardiovascular test and Combogene™-brand diagnostic test can also be combined with other genetic analyses indicative of a susceptibility to disease, including those identified on the Online Mendelian Inheritance in Man (OMIM) Morbid Map at www.ncbi.nlm.nih.gov/Omim/getmorbid.cgi (incorporated herein in its entirety). For example, genetic analyses indicative of a susceptibility to breast cancer, including genetic analyses of polymorphisms in the BRCA1 gene (see, for example, www.ncbi.nlm.nih.gov/entrez/dispomim.cgi?id=113705, incorporated herein in its entirety, and in particular the selected allelic variants described therein), genetic analyses of polymorphisms in the BRCA2 gene (see, for example, www.ncbi.nlm.nih.gov/entrez/dispomim.cgi?cmd=entry&id=600185, incorporated herein in its entirety, and in particular the selected allelic variants described therein), and genetic analyses of polymorphisms in the BRCA3 gene (see, for example, www.ncbi.nlm.nih.gov/entrez/dispomim.cgi?id=605365, incorporated herein in its entirety); and genetic analyses indicative of a susceptibility to Wilm's tumour, including for example, genetic analyses of polymorphisms in the WT1 gene (see, for example, www.ncbi.nlm.nih.gov/entrez/dispomim.cgi?id=607102, incorporated herein in its entirety, and in particular the selected allelic variants described therein), can be combined with one of more of the Emphagene™-brand pulmonary test, Respirogene™-brand pulmonary test, Bronchogene™-brand lung cancer test, Cardiogene™-brand cardiovascular test and Combogene™-brand diagnostic test.

Data comprising the results of the genetic analysis (or analyses) performed as above, can also be used in combination with other risk factor and/or health criteria. In particular, the methods of the invention can additionally have regard to risk factors and/or biometric or biomedical parameters, including but not limited to age, sex, familial history, smoking, alcohol consumption, diet, exercise, blood pressure, body weight, body-mass-index, body fat, serum cholesterol and triglyceride levels or ratios including total cholesterol level, high density cholesterol level, ratio of total cholesterol level to high density cholesterol level, low density cholesterol level, hemoglobin A1c score, glucose level, gamma glutamyltransferase level, and other health risk factors.

Further examples of biomedical parameters used in the methods of the invention assess vital organ function, including, for example, serum concentration of at least one of glucose, blood urea nitrogen, creatinine, uric acid, bilirubin, serum glutamic-oxaloacetic transaminase enzyme, serum glutamate pyruvate transaminase enzyme, alkaline phosphatase, lactic acid dehydrogenase, total protein, albumin, globulin, iron, calcium, phosphorous, sodium, potassium, chloride, high density lipoprotein, triglycerides, total cholesterol, very low density lipoprotein, and/or low density lipoprotein. Therapeutic ratios can also be calculated, including, for example, albumin/globulin ratio, total cholesterol/high density lipoprotein ratio, and/or low density lipoprotein/high density lipoprotein ratio.

Further, a health risk factor and/or biometric or biomedical parameter can be evaluated in comparison to a medical index of normal range.

In another embodiment, the invention provides a method of determining the suitability of a subject for an intervention diagnostic of or therapeutic for at least one disease. The first step of the method is to receive data predictive of the predisposition of a subject to one or more diseases or conditions, the data consisting of or including the results of at least one genetic analysis conducted with respect to the diseases or conditions in question.

In other embodiments, the invention provides a system for determining the suitability of a subject for an intervention diagnostic of or therapeutic for at least one disease or condition, said system including:

computer processor means for receiving, processing and communicating data;

storage means for storing data including a reference genetic database of the results of genetic analysis with respect to at least one disease or condition and optionally a reference intervention database of non-genetic risk factors for at least one disease or condition and optionally other terms and conditions upon which an intervention can be made available with respect to said at least one disease or condition; and

a computer program embedded within the computer processor which, once data consisting of or including the result of a genetic analysis for which data is included in the reference genetic database is received, processes said data in the context of said reference databases to determine, as an outcome, whether said intervention should be available, said outcome being communicable once known, preferably to a user having input said data.

In one embodiment, the data can be input by a representative of an intervention provider, preferably a healthcare provider.

In another embodiment, the data can be input by a subject seeking an intervention, their medical advisor or other representative.

Preferably, said system can be accessible via the internet or by personal computer.

Preferably, said reference genetic database includes the results of a disease-associated genetic analysis selected from one or more of the genetic analyses described herein, or one of more of the Emphagene™-brand pulmonary test, Resipirogene, Bronchogene™-brand lung cancer test, Cardiogene™-brand cardiovascular test and Combogene™-brand diagnostic test.

More preferably, said reference genetic database includes the results of all of the Emphagene™-brand pulmonary test, Respirogene™-brand pulmonary test, Bronchogene™-brand lung cancer test, Cardiogene™-brand cardiovascular test and Combogene™-brand diagnostic test.

In yet a further aspect, the invention provides a computer program suitable for use in a system as defined above comprising a computer usable medium having program code embodied in the medium for causing the computer program to process received data consisting of or including the result of at least one disease-associated genetic analysis in the context of both a reference genetic database of the results of said at least one disease-associated genetic analysis and optionally a reference intervention database of non-genetic risk factors for at least one disease or condition and optionally other terms and conditions upon which an intervention with respect to said at least one disease-associated genetic analysis can be made available.

Preferably, the at least one disease-associated genetic analysis is selected from the Emphagene™-brand pulmonary test, Respirogene™-brand pulmonary test, Bronchogene™-brand lung cancer test, Cardiogene™-brand cardiovascular test and Combogene™-brand diagnostic test.

In a still further aspect, the invention provides for the use of data predictive of the predisposition of a subject to at least two diseases or conditions, at least one of which is selected from Chronic obstructive pulmonary disease (COPD), emphysema, Occupational chronic obstructive pulmonary disease (OCOPD), lung cancer or Acute coronary syndrome (ACS), in the determination of the suitability of a subject for an intervention diagnostic of or therapeutic for at least one of the at least two diseases or conditions,

said data consisting of or including the result of at least one genetic analysis selected from the Emphagene™-brand pulmonary test (as herein defined), the Respirogene™-brand pulmonary test (as herein defined), the Bronchogene™-brand lung cancer test (as herein defined), the Cardiogene™-brand cardiovascular test (as herein defined) or the Combogene™-brand diagnostic test (as herein defined), and said data being representative of the subject's suitability for an intervention diagnostic of or therapeutic for at least one of the at least two diseases or conditions. As discussed above, an increasing number of diseases or conditions are believed to have a genetic component. This can be associated with disease onset, duration, severity, recurrence, and the like. As our understanding of the etiology of a given disease or condition improves, it is likely more and more markers associated with predisposition to that disease or condition will be found. Any disease or condition in which a genetic marker such as a polymorphism can be associated with decreased predisposition (herein “a protective polymorphism”) and/or increased predisposition (herein “a susceptibility polymorphism”) to the disease or condition is amenable to use in the methods of the present invention.

Examples of such diseases which are particularly relevant to the present invention, are given below.

Chronic Obstructive Pulmonary Disease

Chronic obstructive pulmonary disease (COPD) is the 4^{th} leading cause of death in developed countries and a major cause for hospital readmission world-wide. It is characterised by insidious inflammation and progressive lung destruction. It becomes clinically evident after exertional breathlessness is noted by affected smokers when 50% or more of lung function has already been irreversibly lost. This loss of lung function is detected clinically by reduced expiratory flow rates (specifically forced expiratory volume in one second or FEV1). Over 95% of COPD is attributed to cigarette smoking yet only 20% or so of smokers develop COPD (herein termed susceptible smokers). Studies surprisingly show that smoking dose accounts for only about 16% of the impaired lung function.

COPD is a heterogeneous disease encompassing, to varying degrees, emphysema and chronic bronchitis which develop as part of a remodelling process following the inflammatory insult from chronic tobacco smoke exposure and other air pollutants. A number of family studies comparing concordance in siblings (twins and non-twin) consistently show a strong familial tendency. It is likely that many genes are involved in the development of COPD.

Despite advances in the treatment of airways disease, current therapies do not significantly alter the natural history of COPD with progressive loss of lung function causing respiratory failure and death. Although cessation of smoking has been shown to reduce this decline in lung function if this is not achieved within the first 20 years or so of smoking for susceptible smokers, the loss is considerable and symptoms of worsening breathlessness cannot be averted. A number of epidemiology studies have consistently shown that at exposure doses of 20 or more pack years, the distribution in lung function tends toward trimodality with a proportion of smokers maintaining normal lung function (resistant smokers) even after 60+ pack years, a proportion showing modest reductions in lung function who can never develop symptoms and a proportion who show an accelerated loss in lung function who invariably develop COPD. This suggests that amongst smokers 3 populations exist, those resistant to developing COPD, those at modest risk and those at higher risk (termed susceptible smokers).

Therefore, when considering a decision relating to the health of a subject, particularly whether or not the subject is suitable for an intervention, it would be advantageous to be able to identify resistant smokers, those at moderate risk, and those smokers who are most susceptible to developing COPD. For example, it would be advantageous to be able to determine if a given subject was resistant to, at moderate risk of, or susceptible to developing COPD, and in one particularly preferred example, if a smoker previously believed to be susceptible to COPD is determined to be resistant to developing COPD.

Methods to determine a subject's predisposition to and/or potential risk of developing chronic obstructive pulmonary disease (COPD) and/or emphysema are described in New Zealand Patent Application No. 539934, No. 541935, No. 545283, and PCT International Application PCT/NZ2006/000103 (published as WO2006/121351) each incorporated herein in its entirety, and are referred to collectively herein as the Emphagene™-brand pulmonary test. Both protective polymorphisms and susceptibility polymorphisms have been identified for analysis as part of the Emphagene™-brand pulmonary test.

Occupational Chronic Obstructive Pulmonary Disease

Occupational chronic obstructive pulmonary disease (OCOPD) is a well-recognized and well-studied consequence of chronic exposure to a diverse range or aero-pollutants in the workplace. A recent document published by the American Thoracic Society on the occupational contribution to COPD estimates that 15% of all COPD is work related with annual costs of US $7 billion [see 1]. OCOPD is ranked the second highest cause of occupationally related death and believed to be on the rise.

Both cross sectional and prospective studies have shown that OCOPD occurs in a range of occupations characterized by chronic exposure to dust and/or other aero-pollutants including organic and inorganic aero-pollutants. These occupations and industries include metallurgy, iron and steel workers, wood processing workers, chemistry and chemical workers, pulp and paper manufacturing, printing industry, farmers, armed forces, flour milling, popcorn manufacturing, coal, gold, silica and rock miners, welders, painters, boat builders, cotton/synthetic textile workers, construction workers, tobacco workers, and ammonia workers. Examples of pollutants associated with OCOPD include heavy metals (including Cadmium and Vanadium), Nitrogen dioxide, Sulphur dioxide, grain dust, endotoxin, solvents and resins.

In two separate studies, it is estimated that around 40 million people in the United States work force are employed in the “at risk” occupations listed above [see 2, 3].

Studies show that OCOPD results from host factors (including genetic makeup) in combination with exposure dose (for example, concentration and duration). It has been estimated that about 20% of those workers in these occupations can be susceptible to OCOPD.

Importantly, the link between the above occupations and risk of OCOPD is independent of the effects of smoking, ethnicity, and age. In nonsmokers it has been shown that the effect from repeated exposure to the dusts or fumes from the above occupations is equivalent to the effect of smoking in inducing COPD. Moreover, for smokers the combined effect of their smoking and occupational exposure on decline in lung function is greater than either one alone. Therefore, smokers who are also exposed to aero-pollutants at work are at significant risk.

OCOPD is characterised by insidious inflammation and progressive lung destruction. It becomes clinically evident after exertional breathlessness is noted by affected subjects when 50% or more of lung function has already been irreversibly lost. This loss of lung function is detected clinically by reduced expiratory flow rates (specifically forced expiratory volume in one second or FEV1).

Despite advances in the treatment of airways disease, current therapies do not significantly alter the natural history of OCOPD with progressive loss of lung function causing respiratory failure and death. Although cessation of occupational exposure can be expected to reduce this decline in lung function, it is probable that if this is not achieved at an early stage, the loss is considerable and symptoms of worsening breathlessness likely cannot be averted.

Therefore, when considering a decision relating to the health of a subject, particularly whether or not the subject is suitable for an intervention, it would be advantageous to be able to identify resistant subjects and those subjects who are susceptible to developing OCOPD. For example, it would be advantageous to be able to determine if a given subject was resistant to or susceptible to developing OCOPD, and in one particularly preferred example, if a subject previously believed to be susceptible to OCOPD is determined to be resistant to developing OCOPD.

Methods to determine a subject's predisposition to and/or potential risk of developing occupational chronic obstructive pulmonary disease (OCOPD) are described in New Zealand Patent Application No. 540202, No. 541389, and PCT International Application PCT/NZ2006/000124 (published as WO2006/123954) each incorporated herein in its entirety, and are referred to collectively herein as the Respirogene™-brand pulmonary test. Both protective polymorphisms and susceptibility polymorphisms have been identified for analysis of part of the Respirogene™-brand pulmonary test.

Acute Coronary Syndrome

The group of cardiovascular disorders herein referred to as acute coronary syndrome (ACS) includes myocardial infarction and unstable angina. These disorders are believed to be associated with inflammation, plaque instability, and/or smoking. The Applicants believe, without wishing to be bound by any theory, that genetic risk factors are significant in susceptibility to and/or severity of ACS.

Therefore, when considering a decision relating to the health of a subject, particularly whether or not the subject is suitable for an intervention, it would be advantageous to be able to identify resistant subjects and those subjects who are susceptible to developing ACS. For example, it would be advantageous to be able to determine if a given subject was resistant to or susceptible to developing ACS, and in one particularly preferred example, if a subject previously believed to be susceptible to ACS is determined to be resistant to developing ACS.

Methods to determine a subject's predisposition to and/or potential risk of developing ACS are described in New Zealand Patent Application No. 543520, No. 543985, No. 549951, and PCT International Application PCT/NZ2006/000292 each incorporated herein in its entirety, and are referred to collectively herein as the Cardiogene™-brand cardiovascular test.

Lung Cancer

Lung cancer is the second most common cancer and has been attributed primarily to cigarette smoking. Other factors contributing to the development of lung cancer include occupational exposure, genetic factors, radon exposure, exposure to other aero-pollutants and possibly dietary factors [see 4]. Non-smokers are estimated to have a one in 400 risk of lung cancer (0.25%). Smoking increases this risk by approximately 40 fold, such that smokers have a one in 10 risk of lung cancer (10%) and in long-term smokers the life-time risk of lung cancer has been reported to be as high 10-15% [see 5]. Genetic factors are thought to play some part as evidenced by a weak familial tendency (among smokers) and the fact that only the minority of smokers get lung cancer. It is generally accepted that the majority of this genetic tendency comes from low penetrant high frequency polymorphisms, that is, polymorphisms which are common in the general population that in context of chronic smoking exposure contribute collectively to cancer development [see 5, 6]. Several epidemiological studies have reported that impaired lung function [see 7-11] or symptoms of obstructive lung disease [see 12] are independent risk factors for lung cancer and are possibly more relevant than smoking exposure dose.

Despite advances in the treatment of airways disease, current therapies do not significantly alter the natural history of lung cancer, which can include metastasis and progressive loss of lung function causing respiratory failure and death. Although cessation of smoking can be expected to reduce this decline in lung function, it is probable that if this is not achieved at an early stage, the loss is considerable and symptoms of worsening breathlessness likely cannot be averted. The early diagnosis of lung cancer or of a propensity to developing lung cancer enables a broader range of prophylactic or therapeutic treatments to be employed than can be employed in the treatment of late stage lung cancer. Such prophylactic or early therapeutic treatment is also more likely to be successful, achieve remission, improve quality of life, and/or increase lifespan.

Therefore, when considering a decision relating to the health of a subject, particularly whether or not the subject is suitable for an intervention, it would be advantageous to be able to identify resistant subjects and those subjects who are susceptible to developing lung cancer. For example, it would be advantageous to be able to determine if a given subject was resistant to or susceptible to developing lung cancer, and in one particularly preferred example, if a subject previously believed to be susceptible to lung cancer is determined to be resistant to developing lung cancer.

Methods to determine a subject's predisposition to and/or potential risk of developing lung cancer are described in New Zealand Patent Applications No. 540203, No. 541787, No. 543297, No. 550643, No. 554707, and PCT International Application PCT/NZ2006/000125 (published as WO2006/123955) each incorporated herein in its entirety, and are referred to collectively herein as the Bronchogene™-brand lung cancer test. Both protective polymorphisms and susceptibility polymorphisms have been identified for analysis as part of the Bronchogene™-brand lung cancer test.

Combogene

The methods of the present invention can utilise as a genetic analysis the methods of deriving a net score predictive of a subject's predisposition to a disease or condition, for example, as defined in New Zealand Patent Applications No. 540249, No. 541842, No. 551534, and PCT International Application PCT/NZ2006/000104 (published as WO2006/123943). The net score represents the balance between the combined value of the protective polymorphisms present in said subject and the combined value of the susceptibility polymorphisms present in said subject, wherein a net protective score is predictive of a reduced predisposition and/or susceptibility to said disease or condition and a net susceptibility score is predictive of an increased predisposition and/or susceptibility to said disease or condition.

Therefore, when considering a decision relating to the health of a subject, particularly whether or not the subject is suitable for an intervention, it would be advantageous to be able to identify resistant subjects and those subjects who are susceptible to developing one or more diseases or conditions. For example, it would be advantageous to be able to determine if a given subject was resistant to or susceptible to developing a given disease or condition, and in one particularly preferred example, if a subject previously believed to be susceptible to a given disease or condition is determined to have a net protective score and so be resistant to developing said disease or condition.

Methods to determine a subject's net scores are described in New Zealand Patent Applications No. 540249, No. 541842, No. 551534, and PCT International Application PCT/NZ2006/000104 (published as WO2006/123943) each incorporated herein in its entirety, and are referred to collectively herein as Combogene™-brand diagnostic test.

A subject's net score can be placed upon a distribution of net scores for disease sufferers and non-sufferers wherein the net scores for disease sufferers and non-sufferers are or have been determined in the same manner as the net score determined for the subject. By observing where the net score for the subject lies on this distribution, it is possible to identify those subjects having an advantageous risk profile. For example, an health care provider can set a threshold value on said distribution which separates those to whom an intervention will be offered from those to whom an intervention will not be offered. If the net score for a given subject lies within the threshold on said distribution, that subject can be identified as one to whom an intervention can be offered.

As previously indicated, Emphagene™-brand pulmonary test, Respirogene™-brand pulmonary test, Bronchogene™-brand lung cancer test, Cardiogene™-brand cardiovascular test and Combogene™-brand diagnostic test are preferred genetic analyses which can be applied in practising this and other embodiments of this invention.

Armed with the results of the genetic analysis (or analyses), a risk value is determined for the subject. That risk value will be a composite weighting of the data available, with a particular focus on whether the genetic data indicates an increased or reduced predisposition to the diseases tested for.

The risk value is then factored into a health-related decision to be made with respect to the subject. That decision can be made by or for the subject or by a health service provider.

In the case of a health insurer, the decision taken will largely reflect whether the risk value favours the offering of an intervention or not. As one example, should the subject be genetically tested with the results indicative of an increased predisposition to COPD when compared to other subjects of equivalent age, gender and history, the decision can be to offer an intervention therapeutic for COPD to that subject.

Conversely, should the results for the subject be indicative of a reduced predisposition to COPD when compared to other subjects of equivalent age, gender and history, the decision can be to decline to offer the subject an intervention therapeutic for COPD.

Methods of the invention will now be described in more detail, with reference to the following non-limiting representative examples.

### EXAMPLES

### Example 1

### Case Association Study—COPD

As discussed in PCT International Application PCT/NZ2004/000103 (published as WO 2006/121351), a linear relationship between SNP score and frequency of COPD was determined when the polymorphisms shown in Table 1 below were analysed.

Table 1 below presents a summary of the protective and susceptibility SNPs identified in PCT/NZ2004/000103 and related applications. Odd's ratios (OR) and p values are for COPD sufferers compared to resistant smokers with normal lung function. Selected susceptibility SNPs and selected protective SNPs were included in panels of SNPs used to generate a SNP score as discussed below.

TABLE 1
Summary table of protective and susceptibility polymorphisms - COPD

| Gene | Polymorphism | Genotype | Phenotype | OR | P value |
|---|---|---|---|---|---|
| Cyclo-oxygenase 2 (COX2) | −765 G/C¹ | CC/CG | protective | 1.98 | 0.003 |
| β2-adrenoreceptor (ADBR) | Arg16Gly | GG | susceptibility | 1.83 | 0.004 |
| Interleukin-18 (IL18) | −133 C/G | CC | susceptibility | 1.44 | 0.06 |
| Interleukin-18 (IL18) | 105 A/C¹ | AA | susceptibility | 1.50 | 0.04 |
| Plasminogen activator | −675 4G/5G¹ | 5G5G | susceptibility | 1.55 | 0.08 |
| inhibitor 1 (PAI-1) | | | | | |
| Nitric Oxide synthase 3 | Asp 298 Glu² | TT | protective | 2.20 | 0.03 |
| (NOS3) | | | | | |
| Vitamin D Binding Protein | Lys 420 Thr¹ | AA/AC | protective | 1.39 | 0.10 |
| (VDBP) | | | | | |
| Vitamin D Binding Protein | Glu 416 Asp | TT/TG | protective | 1.53 | 0.06 |
| (VDBP) | | | | | |
| Glutathione S Transferase | Ile105Val² | AA | protective | 1.45 | 0.07 |
| (GSTP-1) | | | | | |
| Interferon γ (IFN-γ) | 874 A/T¹ | AA | susceptibility | 1.51 | 0.08 |
| Interleukin-13 (IL13) | Arg 130 Gln | AA | protective | 2.94 | 0.09 |
| Interleukin-13 (IL13) | −1055C/T¹ | TT | susceptibility | 6.03 | 0.03 |
| α1-antitrypsin (α1-AT) | S allele¹ | MS | protective | 2.42 | 0.01 |
| Tissue Necrosis Factor α | +489 G/A | AA/AG | susceptibility | 1.57 | 0.11 |
| TNFα | | GG | protective | | |
| Tissue Necrosis Factor α | −308 G/A | GG | protective | 0.77 | 0.20 |
| TNFα | | AA/AG | susceptibility | | |
| SMAD3 | C89Y AG | AA/AG | protective | 0.26 | 0.07 |
| | | GG | susceptibility | | |
| Intracellular adhesion | E469K A/G | GG | susceptibility | 1.60 | 0.07 |
| molecule 1 (ICAM1) | | | | | |
| Caspase (NOD2) | Gly 881Arg G/C | GC/CC | susceptibility | 3.20 | 0.11 |
| Mannose binding lectin 2 | 161 G/A | GG | protective | 0.53 | 0.003 |
| (MBL2) | | | | | |
| Chymase 1 (CMA1) | −1903 G/A | AA | protective | 0.73 | 0.17 |
| N-Acetyl transferase 2 | Arg 197 Gln G/A | AA | protective | 0.50 | 0.05 |
| (NAT2) | | | | | |
| Interleukin 1B (IL1B) | −511 A/G | GG | susceptibility | 1.30 | 0.17 |
| Microsomal epoxide | Tyr 113 His T/C | TT | susceptibility | 1.50 | 0.06 |
| hydrolase (MEH) | | | | | |
| Microsomal epoxide | His 139 Arg G/A² | GG | protective | 0.64 | 0.23 |
| hydrolase (MEH) | | | | | |
| 5 Lipo-oxygenase (ALOX5) | −366 G/A | AA/AG | protective | 0.60 | 0.12 |
| | | GG | susceptibility | | |
| Heat Shock Protein 70 (HSP | HOM T2437C | CC/CT | susceptibility | 2.00 | 0.002 |
| 70) | | TT | protective | | |
| Chloride Channel Calcium- | +13924 T/A | AA | susceptibility | 1.70 | 0.03 |
| activated 1 (CLCA1) | | | | | |
| Monocyte differentiation | −159 C/T | CC | susceptibility | 1.40 | 0.15 |
| antigen CD-14 | | | | | |
| Elafin | Exon 1 +49 C/T² | CT/TT | protective | 0.70 | 0.24 |
| B2-adrenergic receptor | Gln 27 Glu C/G² | GG | protective | 0.74 | 0.23 |
| (ADBR) | | | | | |
| Matrix metalloproteinase 1 | −1607 1G/2G¹ | 1G1G/1G2G | protective | 0.55 | 0.009 |
| (MMP1) | | | | | |

¹included in both the 9 SNP panel and the 16 SNP panel.
²included in the 9 SNP panel.

As discussed in PCT International Application PCT/NZ2004/000103 (published as WO 2006/121351), a significant difference in frequency of COPD versus resistance was found in those with no protective polymorphisms compared to those with one or more protective genotypes (OR=2.82, P=0.0004, see PCT International Application PCT/NZ2004/000103 referred to above), such that a 2-3 fold increase in COPD in those with 0 protective genotypes was observed.

This example presents an analysis of distributions of SNP scores derived for COPD sufferers and control resistant smokers using the polymorphisms described in Table 1. The SNPs identified above in Table 1, in addition to the Transforming growth factor beta 1 (TGFB1) codon 10 polymorphism (discussed in PCT International Application PCT/NZ02/00106 (published as WO02/0099134)), were included in the 9 SNP panel used to generate a SNP score as discussed below. These 9 SNPs, the Tissue inhibitor of metalloproteinase 3 (TIMP3)-1296 T/C polymorphism and the α1-antitrypsin 1237 G/A polymorphism (each discussed in PCT International Application PCT/NZ02/00106 (published as WO02/0099134)), and the additional 5 SNPs identified in Table 1 above were included in the 16 SNP panel discussed below.

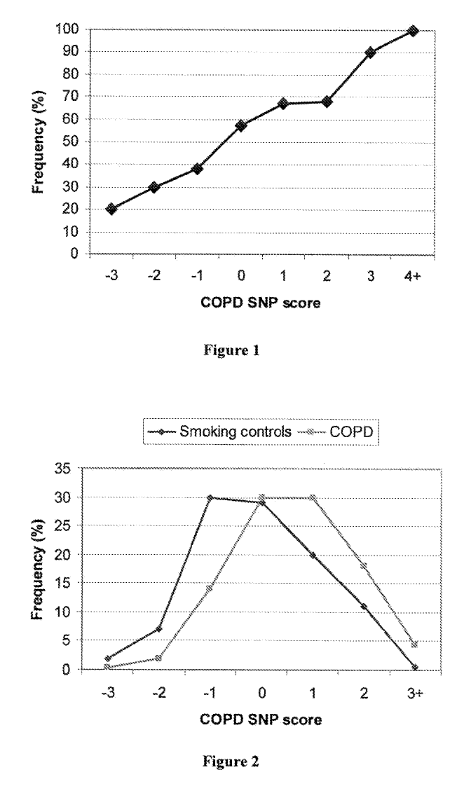

Table 2 below shows the distribution of COPD patients and smoking controls with reference to a SNP score derived from the 9 SNP panel. Each susceptibility SNP was assigned a value of +1, and each protective SNP was assigned a value of −1. The combined scores are added to derive the total SNP score for each subject. The log odds of having ACS plotted against SNP score derived from the 9 SNP panel is shown in FIG. 1, while graphical representation of the distribution shown in Table 2 is shown in FIG. 2.

TABLE 2
Distribution of SNP scores in smokers with and without COPD

| | COPD SNP score - 9 SNP panel | | | | | | | | |
|---|---|---|---|---|---|---|---|---|---|
| Cohort | 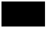 | 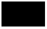 | 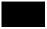 | 0 | 1 | 2 | 3 | 4 | 5 |
| COPDN = 266 | 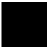 | 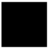 | 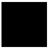 | 79(30%) | 81(30%) | 49(18%) | 9(3%) | 3(1%) | 1(0.4%) |
| controls | 4 | 14 | 61 | 59 | 40 | 23 | 1 | 0 | 0 |
| N = 202 | (2%) | (7%) | (30%) | (29%) | (20%) | (11%) | (0.5%) | (0%) | (0%) |

| | | | | | | | |
|---|---|---|---|---|---|---|---|
| % with | 1/5 | 6/20 | 37/98 | 79/138 | 81/121 | 49/72 | 13/14 |
| COPD | 20% | 30% | 38% | (57%) | (67%) | (68%) | (93%) |

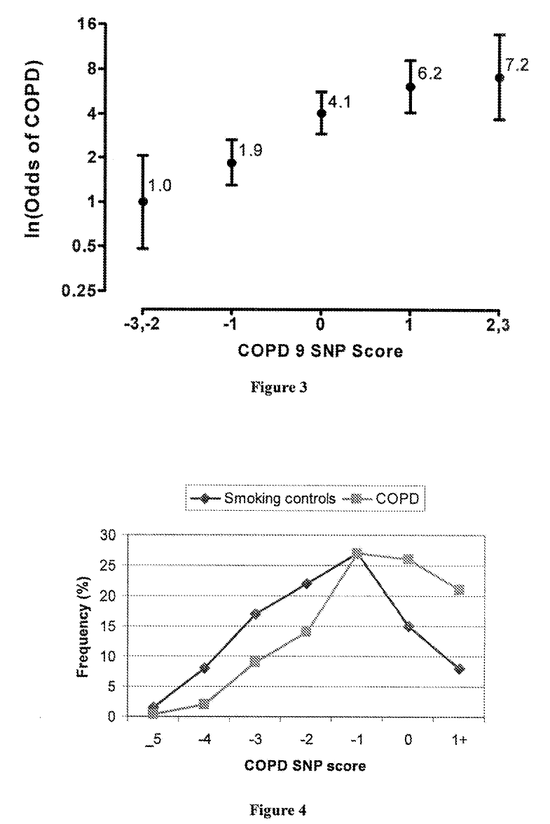

The shaded SNP scores (−3 to −1) can be viewed as low to average risk of COPD. At this cut-off, 16% of COPD sufferers are found and 39% of our control smokers. On the linear figure plotting COPD frequency and SNP score (FIG. 3) this equates to about a 39% risk of COPD.

Table 3 below shows the distribution of COPD patients and smoking controls with reference to a SNP score derived from the 16 SNP panel. Each susceptibility SNP was assigned a value of +1, and each protective SNP was assigned a value of −1. The combined scores are added to derive the total SNP score for each subject. A graphical representation of the distribution shown in Table 3 is shown in FIG. 4.

TABLE 3
Distribution of SNP scores in smokers with and without COPD

| | COPD SNP score - 16 SNP panel | | | | | | |
|---|---|---|---|---|---|---|---|
| Cohort | 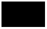 | 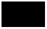 | 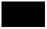 | −2 | −1 | 0 | 1+ |
| COPDN = 266 | 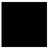 | 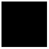 | 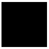 | 37(14%) | 72(27%) | 69(26%) | 56(21%) |
| SmokingcontrolsN = 202 | 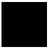 | 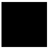 | 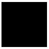 | 44(22%) | 55(27%) | 31(15%) | 1798%) |
| % with COPD | 1/4 | 6/23 | 25/60 | 37/81 | 72/127 | 69/100 | 56/73 |
| | 25% | 26% | 42%) | 46% | 57% | 69% | 77% |

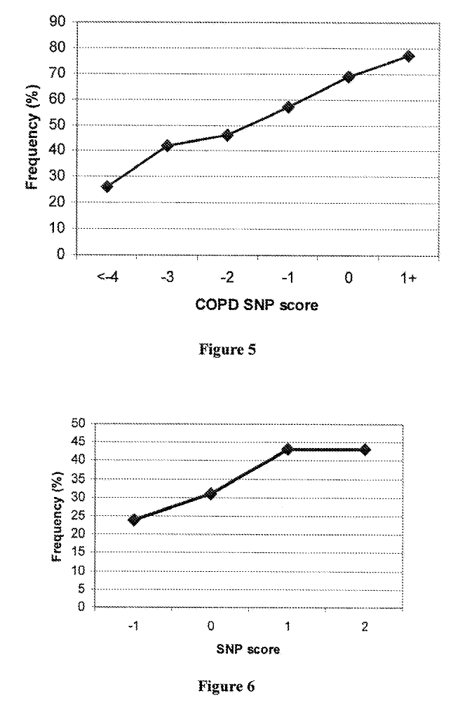

The shaded SNP scores (SNP score ≧−3) can be viewed as low to average risk of COPD. At this cut-off, 11% of COPD sufferers are found and 26.5% of our control smokers. On the linear figure plotting COPD frequency and SNP score (FIG. 5) this equates to about a 20% risk of COPD.

### Example 2

### Case Association Study—Lung Cancer

As discussed in New Zealand Patent Application No.s 540203/541787/543297, New Zealand Patent Application No.s 550643 and 554707, and PCT International application PCT/NZ2006/000125 (published as WO2006/123955), a linear relationship between SNP score and frequency of lung cancer was determined.

Table 4 below presents a summary of the protective and susceptibility SNPs identified in PCT/NZ2006/000125 and related applications, and in New Zealand Patent Application No.s 550643, 554707, and herein.

Statistical Analysis

Patient characteristics in the lung cancer sufferers and controls were compared by unpaired t-tests for continuous variables and chi-square test or Fisher's exact test for discrete variables. Genotype and allele frequencies were checked for Hardy Weinberg Equilibrium and population admixture by the Population structure analysis by genotyping 40 unrelated SNPs. Distortions in the genotype frequencies between lung cancer sufferers and controls were identified using 2 by 3 contingency tables. Where the homozygote genotype (recessive model) or combined homozygote and heterozygote genotypes (codominant model) for the minor allele were found in excess in the healthy smokers controls compared to the lung cancer cohort, these SNP genotypes were assigned as protective. Where the homozygote genotype (recessive model) or combined homozygote and heterozygote genotypes (codominant model) for the minor allele were found in excess in the lung cancer cohort compared to healthy smokers controls, these SNP genotypes were assigned as susceptible. The magnitude of the effect from each SNP was analysed using univariate analysis and multivariate analysis. Based on these analyses, SNPs were ranked according to their ability to discriminate between lung cancer sufferers and controls, and combined as described below to generate a SNP score.

Odd's ratios (OR) and p values are for cancer patients compared to resistant smokers with normal lung function. Selected susceptibility SNPs and selected protective SNPs were included in panels of SNPs to generate a SNP score as discussed below.

TABLE 4
Summary of protective and susceptibility polymorphisms - Lung Cancer

| | | | | | P |
|---|---|---|---|---|---|
| Gene | Polymorphism | Genotype | Phenotype | OR | value |
| Nitric Oxide synthase 3 | Asp 298 Glu | TT | protective | 1.8 | 0.14 |
| (NOS3) | | | | | |
| Nitric Oxide synthase 3 | −786 T/C | TT | susceptibility | 1.4 | 0.23 |
| (NOS3) | | | | | |
| Superoxide dismutase 3 | Arg 312 Gln (+760 | CG/GG | protective | 3.38 | 0.03 |
| (SOD3) | G/C)¹ | | | | |
| XRCC1 | Arg 399 Gln G/A | AA | protective | 2.6 | 0.09 |
| Interleukin-8 (IL-8) | −251 A/T¹ | AA | protective | 4.1 | 0.002 |
| Anti-chymotrypsin | Ala 15 Thr¹ | GG | susceptibility | 1.7 | 0.06 |
| (ACT) | | | | | |
| Cyclin D (CCND1) | A870G | GG | protective | 1.4 | 0.2 |
| | | AA | susceptibility | | |
| Interleukin 1B (IL-1B) | −511 A/G¹ | GG | susceptibility | 1.6 | 0.04 |
| FAS (Apo-1/CD95) | A-670G | AA | susceptibility | 1.5 | 0.15 |
| XPD | Lys −751 Gln G/T¹ | GG | protective | 1.7 | 0.18 |
| CYP 1A1 | Ile 462 Val A/G | GG/AG | protective | 2.2 | 0.12 |
| | | AA | susceptibility | | |
| Matrix metalloproteinase | Asn 357 Ser A/G | GG/AG | protective | 1.7 | 0.23 |
| 12 (MMP12) | | | | | |
| 8-Oxoguanine DNA | Ser 326 Cys G/C | GG | protective | 4.0 | 0.05 |
| glycolase (OGG1) | | | | | |
| N-acetyltransferase 2 | Arg 197 Gln A/G¹ | GG | susceptibility | 1.5 | 0.08 |
| (NAT2) | | | | | |
| CYP2E1 | 1019 G/C (Pst I) | CC/CG | susceptibility | 1.7 | 0.23 |
| CYP2E1 | −1053 C/T (Rsa I)¹ | TT/TC | susceptibility | 1.9 | 0.13 |
| Interleukin-18 (IL-18) | 105 A/C | AC/CC | protective | 1.6 | 0.06 |
| | | AA | susceptibility | | |
| Interleukin-18 (IL-18) | −133 G/C¹ | CG/GG | protective | 1.5 | 0.09 |
| | | CC | susceptibility | | |
| Glutathione S-transferase M | GSTM null | Null | susceptibility | 1.92 | 0.01 |
| Interferon gamma (IFNγ) | 874 A/T | AA | susceptibility | 1.4 | 0.22 |
| Cyclo-oxygenase 2 | −765 G/C | CC/CG | protective | 0.53 | 0.03 |
| (COX2) | | GG | susceptibility | 1.88 | 0.03 |
| Matrix metalloproteinase | −1607 1G/2G | 2G2G | susceptibility | 2.58 | 0.006 |
| 1 (MMP1) | | | | | |
| Connective tissue growth | −447 G/C | GC/CC | susceptibility | 1.6 | 0.19 |
| factor (CTGF) | | | | | |
| Mucin 5AC (MUC5AC) | −221 C/T | TT | protective | 0.47 | 0.14 |
| Mannose binding lectin 2 | +161 G/A | AG/AA | susceptibility | 1.4 | 0.20 |
| (MBL2) | | | | | |
| Nibrin (NBS1) | Gln185Glu G/C | CC | susceptibility | 2.3 | 0.05 |
| Arginase 1 (Arg1) | intron 1 C/T | TT | protective | 0.46 | 0.02 |
| REV1 | Phe 257 Ser C/T¹ | CC | protective | 0.73 | 0.20 |
| Insulin-like growth | Leu252Val C/G | GG | protective | 0.30 | 0.22 |
| factor II receptor | | | | | |
| (IGF2R) | | | | | |
| Apex nuclease (Apex or | Asp148Glu G/T | GG | susceptibility | 1.4 | 0.25 |
| APE1)) | | | | | |
| Interleukin 10 (IL-10) | IL-10 −1082 A/G | GG | protective | 0.66 | 0.15 |
| Cerberus 1 (Cer 1) | R19W A/G² | AA/AG | susceptiblility | 1.7 | 0.02 |
| | (rs 10115703) | | | | |
| XRCC4 | Ser307Ser G/T² | GG/GT | susceptiblility | 1.3 | 0.04 |
| | (rs1056503) | | | | |
| BRCA2 | K3326X A/T² | AT/TT | susceptiblility | 2.5 | 0.04 |
| | (rs 11571833) | | | | |
| Integrin alpha-11 | V433M A/G² | AA | susceptiblility | 4.3 | 0.002 |
| | (rs 2306022) | | | | |
| CAMKK1 | E375G T/C² | TT | protective | 0.76 | 0.13 |
| | (rs7214723) | | | | |
| Tumor protein P73 | −81 C/T | CC | protective | 0.46 | <0.001 |
| (TP73) | (rs2273953)³ | | | | |
| Cytochrome P450 | A/T c74delA³ | AT/TT | susceptiblility | 1.74 | 0.05 |
| polypeptide CYP3A43 | | | | | |
| (CYP3A43) | | | | | |
| B-cell CLL/lymphoma 2 | A/C (rs2279115)³ | AA | protective | 0.69 | 0.05 |
| (BCL2) | | | | | |
| Integrin beta 3 (ITGB3) | A/G +3100 3′UTR | AG/GG | protective | 0.57 | 0.02 |
| | (rs2317676)³ | | | | |
| Dopamine transporter 1 | −3714 G/T | GT/TT | susceptibility | 1.6 | 0.05 |
| (DAT1) | (rs6413429)³ | | | | |
| Tumor necrosis factor | A/G (rs1139417)³ | AA | susceptibility | 1.5 | 0.02 |
| receptor 1 (TNFR1) | | | | | |
| Dopamine receptor D2 | C/Del (rs1799732)³ | CDel/DelDel | protective | 0.61 | 0.02 |
| (DRD2) | | | | | |
| Fas ligand (FasL) | C/T (rs763110)³ | TT | protective | 0.61 | 0.05 |
| Toll-like receptor 9 | C/T (rs5743836)³ | CC | susceptibility | 3.1 | 0.03 |
| (TLR9) | | | | | |

¹included in both the 11 SNP panel and the 16 SNP panel.
²included in both the 5 SNP panel and the 16 SNP panel.
³included in the 9 SNP panel.

This example presents an analysis of distributions of SNP scores derived for lung cancer sufferers and control resistant smokers using the polymorphisms described in Table 4. The SNPs identified in Table 4 by “¹”, in addition to the α1-antitrypsin S allele (AT/TT, susceptibility) and Z allele (AG, protective), each discussed in PCT International application PCT/NZ2006/000125, were included in both the 11 SNP panel and the 16 SNP panel used to generate SNP scores as discussed below. The SNPs identified in Table 4 by “²” were included in both the 5 SNP panel and the 16 SNP panel used to generate SNP scores as discussed below. The SNPs identified in Table 4 by “³”, were included in the 9 SNP panel used to generate SNP scores as discussed below.

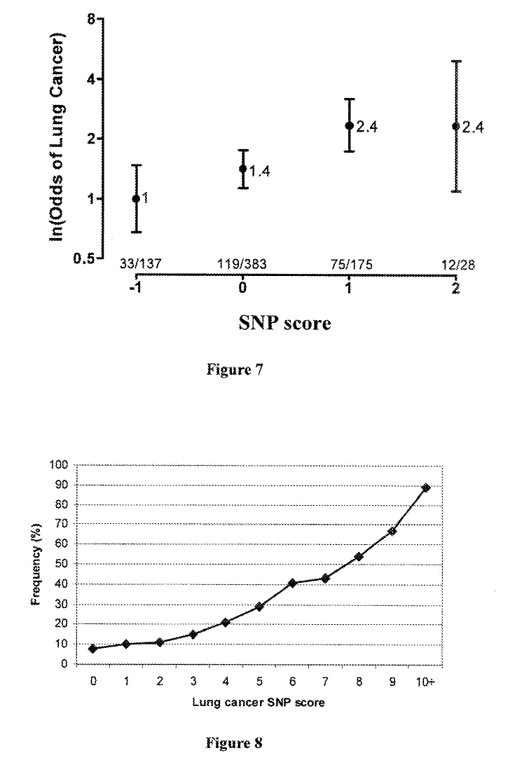

SNP scores for each subject were derived by assigning a score of +1 for the presence of susceptibility genotypes or −1 for the presence of protective genotypes (see Table 4 above). The scores are added to derive the total SNP score for each subject. Table 5 below shows the distribution of SNP scores derived from the 5 SNP panel amongst the lung cancer patients and the resistant smoker controls. The likelihood of having lung cancer according to the lung cancer SNP score is shown graphically in FIG. 6. The log odds of having lung cancer according to the SNP score derived from the 5 SNP panel is shown in FIG. 7.

TABLE 5
Distribution of SNP scores in smokers with and without lung cancer

| | Lung cancer SNP score - 5 SNP panel | | | | | | | |
|---|---|---|---|---|---|---|---|---|
| Cohort | −1 | | 0 | | 1 | | 2 | |
| Lung cancer N = 239 (%) | 33 | (14%) | 119 | (50%) | 75 | (31%) | 12 | (5%) |
| Control smokers N = 484 (%) | 104 | (21%) | 264 | (54%) | 100 | (21%) | 16 | (3%) |
| % with lung cancer | 33/137 | (24%) | 119/383 | (31%) | 75/175 | (43%) | 12/28 | (43%) |

Table 6 below presents the distribution of SNP scores derived from the 11 SNP panel in the lung cancer patients and the resistant smoker controls.

TABLE 6
Distribution of SNP scores in smokers with and without lung cancer

| | Lung cancer SNP score - 11 SNP panel | | | | | | | | | | |
|---|---|---|---|---|---|---|---|---|---|---|---|
| Cohort | 0 | 1 | 2 | 3 | 4 | 5 | 6 | 7 | 8 | 9 | 10+ |
| Lung cancer N = 239 | 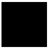 | 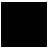 | 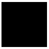 | 12(5%) | 13(5%) | 21(9%) | 47(20%) | 44(18%) | 37(16%) | 24(10%) | 25(10%) |
| Smoking controls N = 484 | 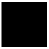 | 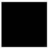 | 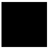 | 69(14%) | 48(10%) | 51(11%) | 68(14%) | 58(12%) | 31(6%) | 14(3%) | 3(1%) |
| % with lung | 6/80 | 7/68 | 8/73 | 15/72 | 26/79 | 37/107 | 37/82 | 44/79 | 29/44 | 16/22 | 14/17 |
| cancer | (8%) | (10%) | (11%) | (21%) | (33%) | (37%) | (45%) | (56%) | (66%) | (73%) | (82%) |

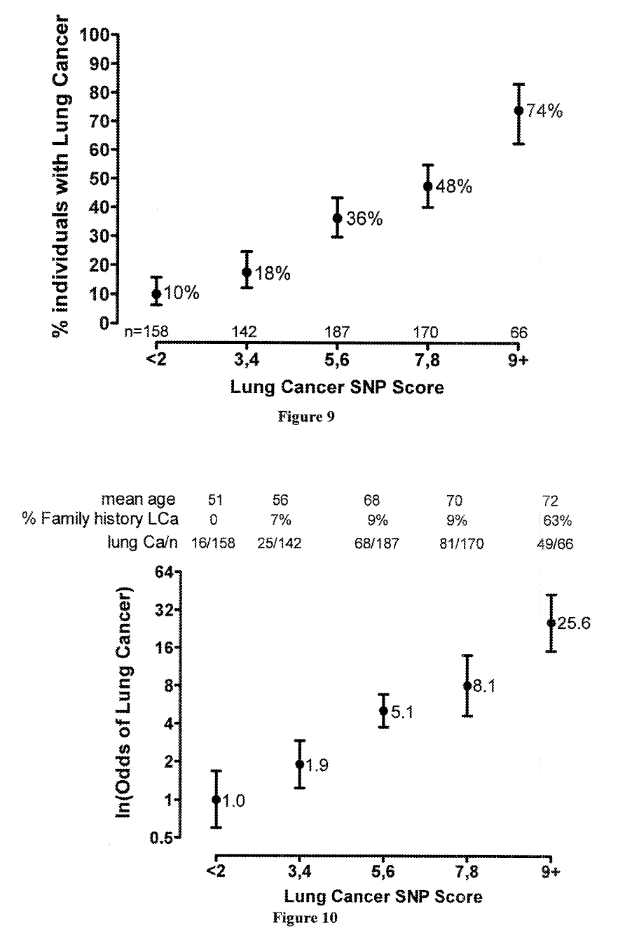

The shaded SNP scores (0 to 2) can be viewed as low to average risk of lung cancer. At this threshold (cut-off), 7% of lung cancer cases were present, while 29% of the control smokers were present. On the graph plotting lung cancer frequency versus SNP score (FIG. 8), this equates to an approximately 10% risk of lung cancer. This is the average across all smokers. The likelihood of having lung cancer according to the SNP score derived from the 11 SNP panel is shown in FIG. 8. The percentage of individuals with lung cancer plotted against SNP score derived from the 11 SNP panel is shown in FIG. 9, while the log odds of having lung cancer plotted against SNP score derived from the 11 SNP panel is shown in FIG. 10.

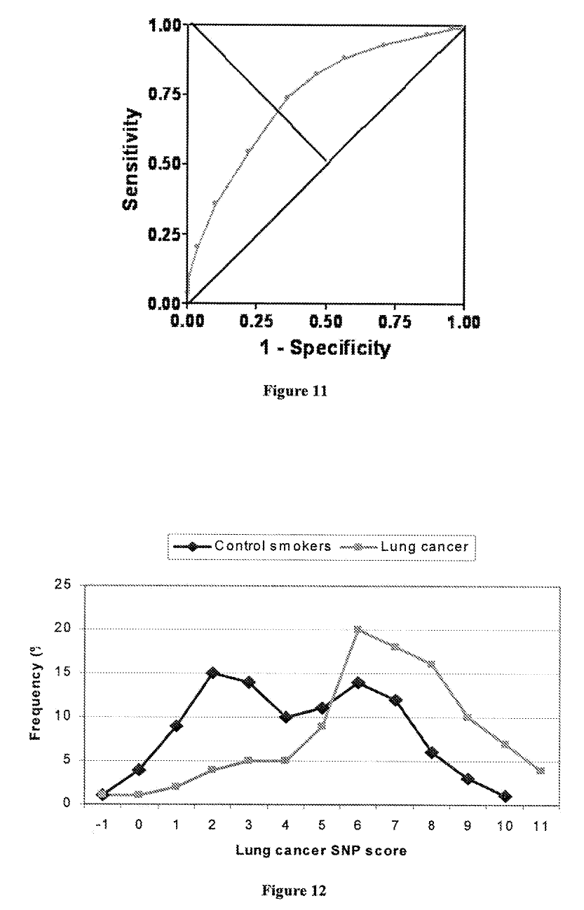

The distribution of SNP scores among lung cancer patients and resistant smoker controls were further analysed as follows. FIG. 11 depicts a receiver-operator curve analysis with sensitivity and sensitivity for the lung cancer 11 SNP panel. This was developed according to the model:

(IL18_{—}133_S+CYP2E1_Rsa1_S+NAT2_{—}197_S+IL1B_{—}511_S+ACT_{—}15_S+s_allele_S+IL8_{—}251_S+z_allele_s)−(XPD_{—}751_P+SOD3_{—}213_P+REV1_{—}257_P)
if age>60 then add 4
if FHx lung Ca then add 3

| Area under the ROC curve | Results |
|---|---|
| Area | 0.7483 |
| Std. Error | 0.01907 |
| 95% confidence interval | 0.7109 to 0.7856 |
| P value | <0.0001 |

| Cutoff | Sensitivity | 95% CI | Specificity | 95% CI | Likelihood ratio |
|---|---|---|---|---|---|
| −0.5000 | 0.9958 | 0.9769 to 0.9999 | 0.004132 | 0.0005008 to 0.01485 | 1.00 |
| 0.5000 | 0.9916 | 0.9701 to 0.9990 | 0.04752 | 0.03036 to 0.07045 | 1.04 |
| 1.500 | 0.9707 | 0.9406 to 0.9881 | 0.1405 | 0.1108 to 0.1747 | 1.13 |
| 2.500 | 0.9331 | 0.8936 to 0.9613 | 0.2934 | 0.2532 to 0.3362 | 1.32 |
| 3.500 | 0.8828 | 0.8351 to 0.9207 | 0.4360 | 0.3913 to 0.4814 | 1.57 |
| 4.500 | 0.8285 | 0.7746 to 0.8740 | 0.5351 | 0.4896 to 0.5803 | 1.78 |
| 5.500 | 0.7406 | 0.6801 to 0.7950 | 0.6405 | 0.5960 to 0.6833 | 2.06 |
| 6.500 | 0.5439 | 0.4785 to 0.6083 | 0.7810 | 0.7415 to 0.8171 | 2.48 |
| 7.500 | 0.3598 | 0.2990 to 0.4242 | 0.9008 | 0.8707 to 0.9260 | 3.63 |
| 8.500 | 0.2050 | 0.1557 to 0.2618 | 0.9649 | 0.9444 to 0.9794 | 5.84 |
| 9.500 | 0.1046 | 0.06884 to 0.1505 | 0.9938 | 0.9820 to 0.9987 | 16.88 |
| 10.50 | 0.03766 | 0.01736 to 0.07028 | 0.9979 | 0.9885 to 0.9999 | 18.23 |
| 11.50 | 0.004184 | 0.0001059 to 0.02309 | 1.000 | 0.9924 to 1.000 | |

As shown in the model above, non-genetic risk factors including age and family history were also analysed, and combined with the SNP score to generate a composite SNP score. FIG. 12 herein presents a graph showing the distribution of SNP score derived from the 11 SNP panel among lung cancer sufferers and among resistant smoker controls.

Table 7 below presents the distribution of SNP scores derived from the 16 SNP panel in the lung cancer patients and the resistant smoker controls.

TABLE 7
Distribution of SNP scores in smokers with and without lung cancer

| | Lung cancer SNP score - 16 SNP panel | | | | | | | | | | |
|---|---|---|---|---|---|---|---|---|---|---|---|
| Cohort | 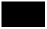 | 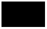 | 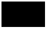 | 4 | 5 | 6 | 7 | 8 | 9 | 10 | 11+ |
| LungcancerN = 239 | 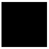 | 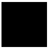 | 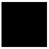 | 15(6%) | 26(11%) | 37(15%) | 37(15%) | 44(18%) | 29(12%) | 16(7%) | 14(6%) |
| SmokingcontrolsN = 484 | 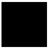 | 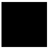 | 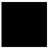 | 57(12%) | 53(11%) | 70(15%) | 45(9%) | 35(7%) | 15(3%) | 6(1%) | 3(1%) |
| % withlungcancer | 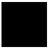 | 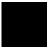 | 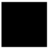 | 15/72(21%) | 26/79(33%) | 37/107(37%) | 37/82(45%) | 44/79(56%) | 29/44(66%) | 16/22(73%) | 14/17(82%) |

The shaded SNP scores (≦1 to 3) can be viewed as low to average risk of lung cancer. At this cut-off, 8% of lung cancer cases were present, while 41% of control smokers were present. On the graph plotting lung cancer frequency and SNP score (FIG. 13), this equates to about a 10% risk of lung cancer, the average across all smokers. The likelihood of having lung cancer according to the SNP score derived from the 16 SNP panel is shown in FIG. 13.

The distribution of SNP scores among lung cancer patients and resistant smoker controls were further analysed as follows. FIG. 14 depicts a receiver-operator curve analysis with sensitivity and sensitivity for the lung cancer 16 SNP panel. This was developed according to the model:

(IL18_{—}133_S+CYP2E1_Rsa1_S+NAT2_{—}197_S+IL1B_{—}511_S+ACT_{—}15_S+s_allele_S+IL8_{—}251_S+z_allele_s)−(XPD_{—}751_P+SOD3_{—}213_P+REV1_{—}257_P)+(ITGA11_s+Cer1_s+BRAC2_s+XRCC4_{—}307_s)−CAMKK1_P
if age>60 then add 4
if FHx lung Ca then add 3

| Area under the ROC curve | Results |
|---|---|
| Area | 0.7621 |
| Std. Error | 0.01855 |
| 95% confidence interval | 0.7257 to 0.7985 |
| P value | <0.0001 |

| Cut off | Sensitivity | 95% Cl | Specificity | 95% Cl | Likelihood ratio |
|---|---|---|---|---|---|
| −0.5000 | 0.9958 | 0.9769 to 0.9999 | 0.01240 | 0.004563 to 0.02679 | 1.01 |
| 0.5000 | 0.9874 | 0.9638 to 0.9974 | 0.05992 | 0.04049 to 0.08492 | 1.05 |
| 1.500 | 0.9749 | 0.9462 to 0.9907 | 0.1529 | 0.1220 to 0.1881 | 1.15 |
| 2.500 | 0.9456 | 0.9088 to 0.9707 | 0.2789 | 0.2394 to 0.3212 | 1.31 |
| 3.500 | 0.9121 | 0.8688 to 0.9448 | 0.4132 | 0.3690 to 0.4585 | 1.55 |
| 4.500 | 0.8494 | 0.7976 to 0.8922 | 0.5310 | 0.4854 to 0.5762 | 1.81 |
| 5.500 | 0.7406 | 0.6801 to 0.7950 | 0.6405 | 0.5960 to 0.6833 | 2.06 |
| 6.500 | 0.5858 | 0.5205 to 0.6489 | 0.7851 | 0.7458 to 0.8209 | 2.73 |
| 7.500 | 0.4310 | 0.3673 to 0.4964 | 0.8781 | 0.8456 to 0.9059 | 3.54 |
| 8.500 | 0.2469 | 0.1935 to 0.3066 | 0.9504 | 0.9271 to 0.9680 | 4.98 |
| 9.500 | 0.1255 | 0.08632 to 0.1743 | 0.9814 | 0.9650 to 0.9915 | 6.75 |
| 10.50 | 0.05858 | 0.03239 to 0.09633 | 0.9938 | 0.9820 to 0.9987 | 9.45 |
| 11.50 | 0.02092 | 0.006827 to 0.04814 | 1.000 | 0.9924 to 1.000 | |

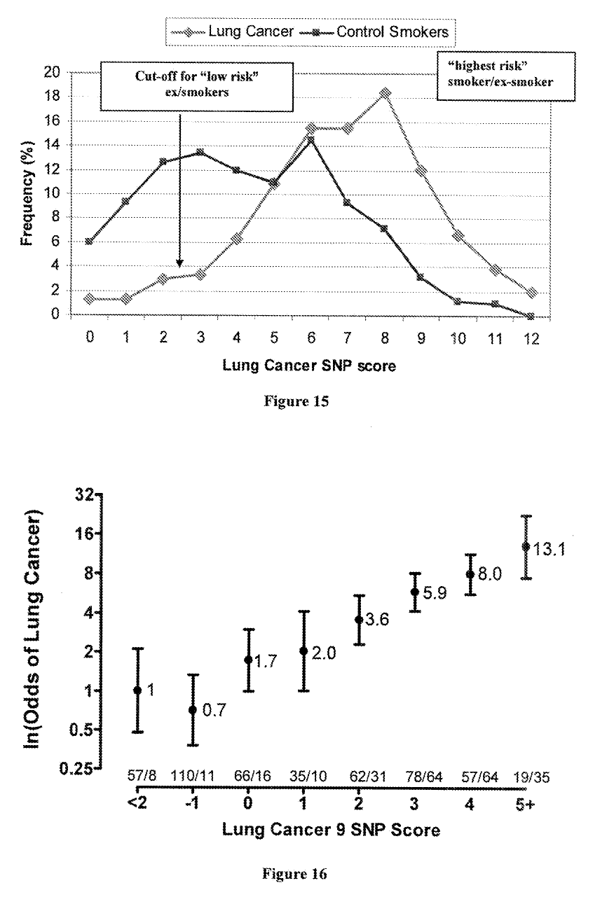

FIG. 15 herein presents a graph showing the distribution of SNP score derived from the 16 SNP panel among lung cancer sufferers and among resistant smoker controls.

A multivariate analysis was performed using a 9 SNP panel comprising the polymorphisms identified in Table 4 above, which summarises the univariate analyses of protective and susceptibility SNPs associated with lung cancer. Odd's ratios (OR) and p values are for cancer patients compared to resistant smokers with normal lung function.

As described above in respect of the 5, 11, and 16 SNP panels, a SNP score was determined for each subject from the univariate data for this 9 SNP panel. The presence of the susceptibility SNP genotype was scored +1, and the presence of the protective SNP genotype was scored −1.

As shown in FIG. 16, a linear relationship was observed when the SNP score for lung cancer patients and healthy smoking controls were analysed together and plotted according to the odds of having lung cancer, where those with the highest scores have the greatest risk. In this analysis (floating absolute odds ratio), the lowest SNP score group is referenced as 1. Those with the highest score (5 or more) have an Odds of 13—that is, they are at 13 fold greater likelihood (or risk) of being diagnosed with lung cancer.

For each subject, a composite score that defines a likelihood of being diagnosed with lung cancer was derived. The SNP score from the 9 SNP panel was combined with scores according to age (+4 for age over 60 years of age) and family history (+3 for having a first degree relative with lung cancer) for each subject. This algorithm generated a composite score for each smoker based on genotype, age and family history of lung cancer. Table 8 below shows the results of this multivariate analysis using these 9 SNPs, age and family history.

TABLE 8
Multivariate analysis

| Analysis of Maximum Likelihood Estimates | | | | | | | | |
|---|---|---|---|---|---|---|---|---|
| | | | | Wald | | | 95% Wald | |
| | | | Standard | Chi- | | | Confidence | |
| Parameter | DF | Estimate | Error | Square | Pr > ChiSq | OR | Limits | |
| Intercept | 1 | 4.1002 | 0.8241 | 24.7553 | <.0001 | | | |
| P73_p | 1 | 0.7646 | 0.1995 | 14.6902 | 0.0001 | 2.148 | 1.453 | 3.176 |
| 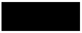 | 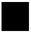 | 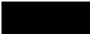 | 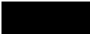 | 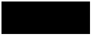 | 0.0142 | 1.910 | 1.139 | 3.204 |
| 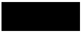 | 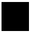 | 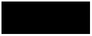 | 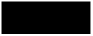 | 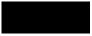 | 0.0960 | 1.469 | 0.934 | 2.310 |
| FasL_p | 1 | 0.8187 | 0.2991 | 7.4906 | 0.0062 | 2.267 | 1.262 | 4.075 |
| ITGB3_p | 1 | 0.7764 | 0.2985 | 6.7636 | 0.0093 | 2.174 | 1.211 | 3.902 |
| TNFR1_s | 1 | −0.1094 | 0.2180 | 0.2517 | 0.6159 | 0.896 | 0.585 | 1.374 |
| CYP3A43_s | 1 | −0.7760 | 0.3741 | 4.3036 | 0.0380 | 0.460 | 0.221 | 0.958 |
| DAT1_s | 1 | −0.4273 | 0.2918 | 2.1431 | 0.1432 | 0.652 | 0.368 | 1.156 |
| TLR9_s | 1 | −0.6429 | 0.6268 | 1.0520 | 0.3050 | 0.526 | 0.154 | 1.796 |
| Age | 1 | −0.0796 | 0.0104 | 58.3869 | <.0001 | 0.923 | 0.905 | 0.943 |
| FHxLCancer | 1 | 0.3105 | 0.2582 | 1.4452 | 0.2293 | 1.364 | 0.822 | 2.263 |
| | | c | 0.770 | | | | | |

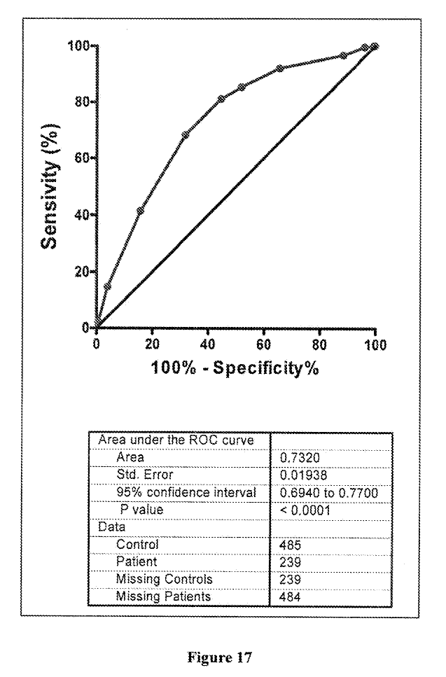

FIG. 17 shows the receiver-operator curve analysis for this composite lung cancer SNP score. The receiver operator curve analysis shows the area under the ROC curve is 0.73 for these 9 SNPs. This indicates an acceptable level of discrimination.

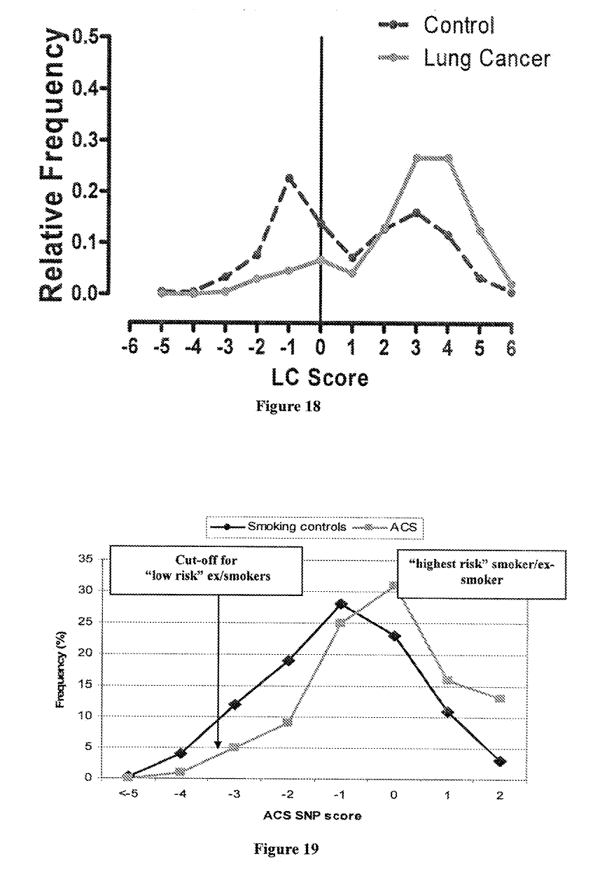

When the frequency distribution for the 9 SNP panel SNP score is compared between lung cancer cases and controls (FIG. 18), separation of the lung cancer SNP score between cases and controls is observed. This reflects the ability of the SNP score to discriminate between high and low risk smokers. This data shows that these SNPs can be analysed in combination to derive a risk score with clinical utility in discriminating smokers at high and low risk of lung cancer based on their genotype, and such analyses can include non-genetic factors such as age and family history.

### Discussion

When the frequency of resistant smokers and smokers with lung cancer were compared according to the SNP score derived from a 5 SNP panel consisting of the SNPs identified in Table 4 herein, the chances of having lung cancer increased from 24%-31% to 43% in smokers with a SNP score of −1, 0, or 1+, respectively. When the frequencies of resistant smokers and smokers with lung cancer were compared according to a SNP score derived from an 11 SNP panel, it was found that the chances of having lung cancer increased from 8% to 82% in smokers with a SNP score of 0 compared to those with a SNP score of 10+.

A minor increase in the linearity of the relationship between SNP score and frequency of lung cancer was observed when the SNP score was derived from a 16 SNP panel consisting of the SNPs identified in Table 4. Again, the chances of having lung cancer increased from 8%, to 82% in smokers with a SNP score of less than or equal to 1 compared to those with a SNP score of 11+. The slight increase in linearity can be seen in a comparison of FIG. 8 (11 SNP panel) and FIG. 13 (16 SNP panel).

When the frequency of resistant smokers and smokers with lung cancer were compared according to the SNP score derived from a 9 SNP panel consisting of the SNPs identified in Table 4 herein, the chance of having lung cancer was increased 13-fold in smokers with a SNP score of 5+ compared to those with a SNP score of 1.

These findings indicate that the methods of the present invention can be predictive of lung cancer in an individual well before symptoms present.

Importantly, a substantial difference is seen in the distribution of lung cancer patients and control smokers relative to total SNP score when the SNP score is derived from the 16 SNP panel rather than from the 11 SNP panel (see FIG. 15 compared to FIG. 12). In this analysis, the addition of the 5 SNPs discussed herein to the 11 SNP panel results in only a small change to the linear relationship between lung cancer SNP score and frequency of lung cancer (see FIGS. 8 and 13), and results in only a small difference to the receiver-operator curve analysis with sensitivity and specificity (See FIGS. 11 and 14), yet results in a substantial difference to the utility of the SNP score, identifying a larger subgroup of control smokers who are “low risk” defined by a cut off over the linear scale of SNP score. A similarly useful discrimination between lung cancer sufferers and resistant controls was observed when a distribution of SNP scores calculated using the 9 SNP panel was derived—see FIG. 18. This has important implications in rationing or prioritising medical interventions.

These findings indicate that the methods of the present invention can be used to identify subsets of nominally at risk individuals (and particularly smokers) who are at low to average risk of lung cancer, and are thus not suitable for an intervention.

These findings therefore also present opportunities for therapeutic interventions and/or treatment regimens, as discussed herein. Briefly, such interventions or regimens can include the provision to the subject of motivation to implement a lifestyle change, or therapeutic methods directed at normalising aberrant gene expression or gene product function. In another example, a given susceptibility genotype is associated with increased expression of a gene relative to that observed with the protective genotype. A suitable therapy in subjects known to possess the susceptibility genotype is the administration of an agent capable of reducing expression of the gene, for example using antisense or RNAi methods. An alternative suitable therapy can be the administration to such a subject of an inhibitor of the gene product. In still another example, a susceptibility genotype present in the promoter of a gene is associated with increased binding of a repressor protein and decreased transcription of the gene. A suitable therapy is the administration of an agent capable of decreasing the level of repressor and/or preventing binding of the repressor, thereby alleviating its downregulatory effect on transcription. An alternative therapy can include gene therapy, for example the introduction of at least one additional copy of the gene having a reduced affinity for repressor binding (for example, a gene copy having a protective genotype).

Suitable methods and agents for use in such therapy are well known in the art, and are discussed herein.

### Example 3

### Case Association Study—ACS

As disclosed in New Zealand Patent Application No. 543520, No. 543985, No. 549951, and PCT International application PCT/NZ2006/000292, a linear relationship between SNP score and frequency of ACS was determined when the polymorphisms shown in Table 9 below were analysed.

Table 9 below presents a summary of the protective and susceptibility SNPs identified in PCT/NZ2006/000292 and related applications. Selected susceptibility SNPs are identified as S1 through S13, while selected protective SNPs are identified as P1 through P16. Those shown in bold were included in panels of SNPs used to generate a SNP score as discussed below.

TABLE 9
Summary of Protective and susceptibility SNPs for ACS

| SNP# | Gene | Polymorphism | Genotype | Phenotype | OR | P value |
|---|---|---|---|---|---|---|
| S1 | CMA1 | −1903 A/G (rs1800875) | GG | susceptibility | 1.9 | 0.004 |
| S2 | TGFB1 | −509 C/T (rs1800469) | CC | susceptibility | 1.5 | 0.05 |
| S3 | MMP12 | −82 A/G (rs2276109) | GG | susceptibility | 3.2 | 0.05 |
| S4 | FGF2 | Ser52Ser 223 C/T | CT/TT | susceptibility | 1.5 | 0.08 |
| | | (rs1449683) | (CC) | (protective) | | |
| S5 | IL4RA | Q576R A/G (rs1801275) | GG | susceptibility | 2.7 | 0.02 |
| | | | AA | protective | 0.47 | 0.05 |
| P1 | LTA | Thr26Asn A/C | CC | protective | 0.66 | 0.04 |
| | | (rs1041981) | | | | |
| P2 | HSP70 | Hom T2437C | CC/CT | protective | 0.66 | 0.04 |
| | | (rs2227956) | (TT) | (susceptibility) | | |
| P3 | TLR4 | ¹Asp299Gly A/G | AG/GG | protective | 0.54 | 0.07 |
| | | (rs4986790) | (AA) | (susceptibility) | | |
| P3.1 | TLR4 | ²Thr399Ile C/T | CT/TT | protective | 0.54 | 0.06 |
| | | (rs4986791) | (CC) | (susceptibility) | | |
| P4 | IFNG | 874 A/T (rs2430561) | TT | protective | 0.57 | 0.03 |
| P11 | NFKBIL1 | −63 T/A (rs2071592) | AA | protective | 0.73 | 0.10 |
| P5 | PDGFRA | −1630 I/D, (AACTT/Del) | I/Del, Del/Del | protective | 0.68 | 0.05 |
| | | | (II) | (susceptibility) | | |
| P6 | IL4 | −589 C/T (rs2243250) | CT/TT | protective | 0.68 | 0.11 |
| | | | (CC) | (susceptibility) | | |
| S6 | MMP1 | −1607 1G/2G (Del/G) | Del.Del (ie | susceptibility | 1.4 | 0.12 |
| | | (rs1799750) | 1G1G) | | | |
| S7 | PDGFA | 12 IN5 C/T | TT | susceptibility | 1.4 | 0.14 |
| S8 | GCLM | −588 C/T | CT/TT | susceptibility | 1.4 | 0.13 |
| | | | (CC) | (protective) | | |
| S9 | OR13G1 | Ile132Val A/G | AA | susceptibility | 1.4 | 0.14 |
| | | (rs1151640) | | | | |
| P12 | IL-10 | −1084 A/G (−1082) | GG | protective | 0.74 | 0.19 |
| | | (rs1800896) | | | | |
| S10 | α1-AT S | Glu288Val A/T (M/S) | AT/TT | susceptibility | 1.5 | 0.16 |
| | allele | (rs17580) | (MS/SS) | | | |
| P7 | ICAM1 | K469E A/G (rs5498) | AA | protective | 0.70 | 0.09 |
| P8 | BAT1 | −23 C/G (rs2239527) | GG | protective | 0.71 | 0.09 |
| P9 | NOS3 | Glu298Asp G/T | GG | protective | 0.72 | 0.09 |
| | | (rs1799983) | | | | |
| P10 | SOD3 | Arg213Gly C/G | CG/GG | protective | 0.23 | 0.13 |
| | | (rs1799895) | | | | |
| P13 | PAI-1 | −668 4G/5G | 5G5G | protective | 0.72 | 0.19 |
| S11 | MIP1A | +459 C/T Intron 1 | CT/TT | susceptibility | 1.31 | 0.18 |
| | | (rs1719134) | | | | |
| P14 | MMP7 | −181 A/G (rs17880821) | GG | protective | 0.70 | 0.19 |
| P15 | Cathepsin G | Asn 125Ser | AG/GG | protective | 0.58 | 0.12 |
| | | | AA | (susceptibility) | | |
| S12 | CX3CR1 | I249V (rs3732379) | TT | susceptibility | 1.5 | 0.15 |
| S13 | NOD2 | Gly 881 Arg G/C | CC/CG | susceptibility | 2.1 | 0.15 |
| | | (rs2066845) | | | | |
| P16 | TIMP1 | 372 T/C (rs4898) | TT | protective | 0.27 | 0.00005 |
| | | | CC | susceptibility | 1.4 | 0.06 |

S3 above is in linkage disequilibrium (LD) with S6, P1 above is in LD with P11 and P3 above is in LD with P3.1. Hence, these SNPs were not used together in a panel when deriving the SNP score.

Table 10 below shows the distribution of ACS patients and smoking controls with reference to a SNP score. The SNP score for each individual was determined in a combined analysis of an 11 SNP panel consisting of SNPs S1-S5 and P1-P6 as shown in Table 9. Each susceptibility SNP was assigned a value of +1, and each protective SNP was assigned a value of −1. The combined scores are added to derive the total SNP score for each subject. FIG. 19 presents this data graphically.

TABLE 10
Distribution of SNP scores in smokers with and without ACS

| | ACS SNP score - 11 SNP panel | | | | | | | |
|---|---|---|---|---|---|---|---|---|
| Cohort | 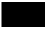 | 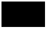 | 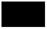 | −2 | −1 | 0 | 1 | 2+ |
| ACS N = 148 | 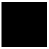 | 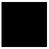 | 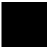 | 13(9%) | 37(25%) | 46(31%) | 24(16%) | 19(13%) |
| Smoking controls | 2 | 16 | 53 | 88 | 129 | 107 | 51 | 14 |
| N = 460 | (0.4%) | (4%) | (12%) | (19%) | (28%) | (23%) | (11%) | (3%) |
| % with ACS | 0/2 | 1/17 | 8/61 | 13/101 | 37/166 | 46/153 | 24/75 | 19/33 |
| | (0%) | (6%) | (13%) | (13%) | (22%) | (30%) | (32%) | (58%) |

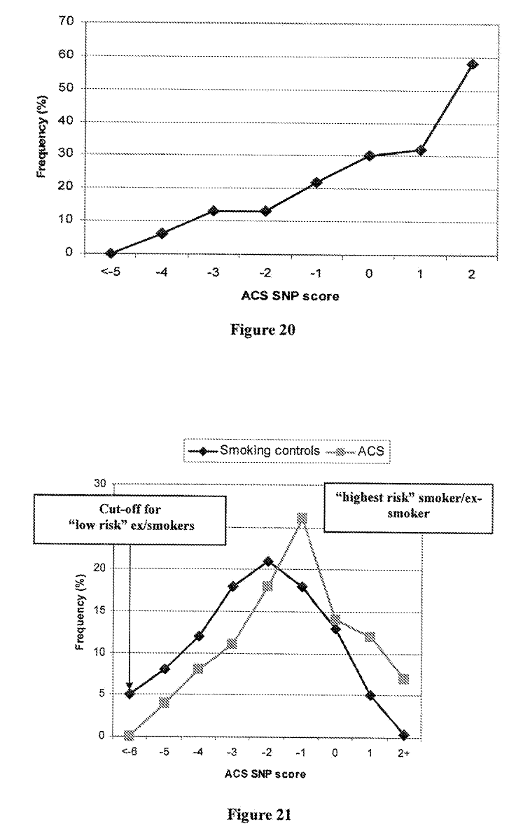

The shaded SNP scores (≦−5 to −2) can be viewed as low to average risk of ACS. At this cut-off, 15% of ACS subjects are found and 35% of control smokers. On the linear figure plotting ACS frequency and SNP score (FIG. 20) this equates to about a 13% risk of ACS.

Table 11 below shows the distribution of ACS patients and smoking controls according to the SNP score determined with reference to a larger, 15 SNP, panel. This 15 SNP panel consisted of SNPs S1-S5 and P1-P10 as shown in Table 9. Again, each susceptibility SNP was assigned a value of +1, and each protective SNP was assigned a value of −1. The combined scores are added to derive the total SNP score for each subject. FIG. 21 presents the data shown in Table 11 graphically.

TABLE 11
Distribution of SNP scores in smokers with and without ACS

| | ACS SNP score - 15 SNP panel | | | | | | | | |
|---|---|---|---|---|---|---|---|---|---|
| Cohort | 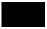 | 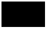 | 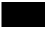 | −3 | −2 | −1 | 0 | 1 | 2+ |
| ACS N = 148 | 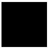 | 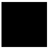 | 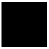 | 16(11%) | 26(18%) | 38(26%) | 21(14%) | 18(12%) | 11(7%) |
| Smoking controls | 22 | 35 | 55 | 84 | 98 | 83 | 60 | 21 | 2 |
| N = 460 | (5%) | (8%) | (12%) | (18%) | (21%) | (18%) | (13%) | (5%) | (0.4%) |
| % with ACS | 0/22 | 6/41 | 12/67 | 16/100 | 26/124 | 38/121 | 21/81 | 18/39 | 11/13 |
| | (0%) | (15%) | (18%) | (16%) | (21%) | (31%) | (26%) | (46%) | (84%) |

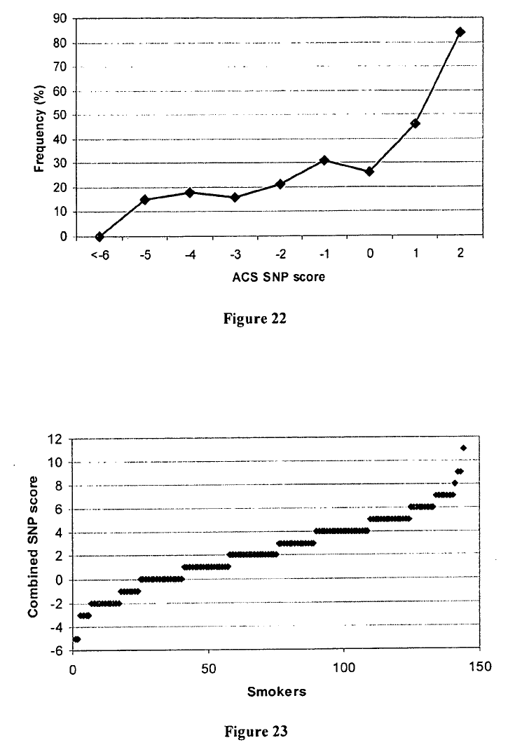

The shaded SNP scores (≦−6 to −4) can be viewed as low to average risk of ACS. At this cut-off, 12% of ACS sufferers are found and 25% of control smokers. On the linear figure plotting ACS frequency and SNP score (FIG. 22) this equates to about a 18% risk of ACS.

### Example 4

### Case Association Study—OCOPD

As discussed in New Zealand Patent Application No. 540202/541389, and PCT International application PCT/NZ2006/000124 (published as WO2006/123954), a linear relationship between SNP score and frequency of OCOPD was determined.

Table 12 below presents a summary of the protective and susceptibility SNPs identified in PCT/NZ2006/000124 and related applications. Selected susceptibility SNPs and selected protective SNPs were included in panels of SNPs used to generate a SNP score as discussed below.

TABLE 12
Summary table of protective and susceptibility polymorphisms - OCOPD

| Gene | Polymorphism | Genotype | Phenotype | OR | P value |
|---|---|---|---|---|---|
| Cyclo-oxygenase 2 (Cox2) | −765 G/C¹ | CC/CG | protective | 2.2 | 0.03 |
| | | GG | susceptibility | 0.5 | 0.03 |
| β2-adrenoreceptor | Gln 27 Glu | CC | protective | 1.75 | 0.05 |
| (ADRB2) | | | | | |
| Interleukin-18 (IL-18) | −133 C/G | CC | susceptibility | 1.8 | 0.04 |
| Interleukin-18 (IL-18) | 105 A/C | AA | susceptibility | 1.8 | 0.05 |
| Plasminogen activator | −675 4G/5G¹ | 5G5G | susceptibility | 1.9 | 0.08 |
| inhibitor 1 (PAI-1) | | | | | |
| Nitric Oxide synthase 3 | Asp 298 Glu¹ | TT | protective | 2.3 | 0.05 |
| (NOS3) | | | | | |
| Vitamin D Binding Protein | Lys 420 Thr¹ | AA | protective | 3.2 | 0.05 |
| (VDBP) | | CC | susceptibility | 1.8 | 0.04 |
| Vitamin D Binding Protein | Glu 416 Asp¹ | TT/TG | protective | 1.9 | 0.04 |
| (VDBP) | | GG | susceptibility | 0.5 | 0.04 |
| Glutathione S Transferase | Ile 105 Val¹ | GG | susceptibility | 2.3 | 0.09 |
| (GSTP1) | | | | | |
| Superoxide dismutase 3 | Arg 312 Gln¹ | AG/GG | protective | 10.8 | 0.01 |
| (SOD3) | | AA | susceptibility | 10.8 | 0.01 |
| α1-antitrypsin (α1AT) | 3′ 1237 G/A (T/t) | Tt/tt | susceptibility | 3.34 | 0.01 |
| α1-antitrypsin (α1AT) | S allele¹ | MS | protective | 2.7 | 0.07 |
| Toll-like receptor 4 (TLR4) | Asp 299 Gly A/G | AG/GG | protective | 5.61 | 0.10 |
| Interleukin-8 (IL-8) | −251 A/T | AA | protective | 1.8 | 0.09 |
| Interleukin 11 (IL-11) | −518 G/A | AA | protective | 1.6 | 0.16 |
| Microsomal epoxide | Exon 3 T/C (r/R)¹ | RR | protective | 2.3 | 0.05 |
| hydrolase (MEH) | | | | | |
| Interleukin 13 (IL-13) | −1055 C/T¹ | TT | susceptibility | 6.03 | 0.03 |
| Matrix Metalloproteinase 1 | −1607 1G/2G¹ | 2G2G | susceptibility | 2.1 | 0.02 |
| (MMP1) | | | | | |

¹included in the 11 SNP panel.

The SNP score for each individual was determined in a combined analysis of the selected protective and susceptibility polymorphisms identified in Table 12 above. Each susceptibility SNP was assigned a value of −1, and each protective SNP was assigned a value of +1. Values were added to derive a net SNP score for the 11 SNP panel. Table 13 below shows the distribution of OCOPD patients and smoking controls with reference to the net SNP score.

TABLE 13
Distribution of SNP scores in exposed smokers
with and without OCOPD

| | OCOPD SNP score - 11 SNP panel | | | | | |
|---|---|---|---|---|---|---|
| Cohort | −2 | −1 | 0 | 1 | 2 | 3 |
| OCOPD n = 124 | 8 | 28 | 33 | 39 | 11 | 5 |
| Exposed Resistant n = 101 | 2 | 11 | 23 | 27 | 23 | 15 |
| % OCOPD | 80% | 72% | 59% | 59% | 32% | 25% |

As shown in Table 13, there was a linear relationship in frequency of OCOPD compared to SNP score in the range of SNP scores from −2 to +3. For subjects with a net score of −1 or less, there was an approximately 70% or greater risk of having OCOPD. In contrast, for subjects with a net score of >+2, the risk was approximately 25%.

Discussion

On the basis of this analysis, SNP scores below 3 are viewed by the health care provider as representing a high risk of OCOPD. Below this threshold, more than 25% of subjects have OCOPD. Subjects with SNP scores below 3 are identified by the health care provider as being suitable for an intervention.

### Example 5

### Case Association Study—Diabetes

Both type 1 and type 2 diabetes are believed to result from the combination of many genetic factors and environmental factors (for example, viral illness with initiation of autoimmunity for type 1 diabetes, and obesity with associated insulin resistance in type 2 diabetes). Genetic variants (polymorphisms) that confer a degree of susceptibility to and protection from diabetes can be identified through family/pedigree based approaches (e.g. linkage analysis, trios, affected sib-pair or transmission disequilibrium tests) or through unrelated individuals in either case-control studies or cohort studies. Each genetic variant can contribute independently to the score in a weighted or unweighted analysis to derive a net score based on an algorithm. Algorithms such as those described herein, where a value of +1 for the presence of a susceptibility genotype at a specific SNP, −1 for the presence of a protective genetic variant, and 0 when neither is present, is assigned, can be used. The total composite score is derived by adding each individual score.

When the distributions of the genetic score versus disease frequency is plotted for the diabetes sufferers and the controls (SNP score on the horizontal axis), it can be possible to show a diverging or bimodal distribution amongst these two groups. The greater the separation, the greater the discrimination between affected and unaffected individuals based on SNP score. Therefore, the better the separation between these distributions, the greater the ability to define a threshold value that defines all (or the majority) of diabetes cases while minimizing the number of controls. Alternatively, a cut off in the SNP score can be identified that will maximize the number of cases identified (i.e., prevalence) in the cohort of people tested. This might be used to maximize the number of people affected in a prospective study. This type of analysis is another way of defining a cut off (threshold) to optimize either sensitivity or specificity according to clinical need.

Summary: Genetic polymorphisms can be combined in algorithms to derive a composite score for diabetes risk where risk conferring polymorphisms are found and when the correct combination of SNPs are analysed. When the frequency of the genetic score for cases and controls are plotted according to distribution, and significant divergence is demonstrated, it is possible to assess the utility of the genetic score for prioritizing at risk people (i.e. segmentation) for population based interventions such as screening. This is an important approach to defining cut-offs at which a genetic score can confer the greatest segmentation for assessing considerations such as cost-effectiveness of various intervention regimes. Such an approach is generalisable to other diseases where the above analysis can be achieved.

### Example 6

### Combined Risk Assessment

This example recognizes studies reporting that 50% of smokers die from their smoking and 25% die before aged 65 years of age. Of those that die prematurely, 80% of deaths are attributed to coronary artery disease, lung cancer and COPD. The Applicant's believe that a smoker's susceptibility to these diseases are in part due to genetic predisposition, and that if this predisposition could be identified, smokers could be identified at a young age and through genotyping determine who are low, medium and high risk for these conditions.

144 volunteer smokers were genotyped using each of the Emphagene™-brand pulmonary test, the Bronchogene™-brand lung cancer test, and the Cardiogene™-brand cardiovascular test SNP-based tests as described herein to determine the distribution of those smokers that were at high and low risk across all 3 tests. Smokers had a minimum 15 pack year history, and were not diagnosed as ACS, lung cancer or COPD sufferers.

A SNP score for each of the tests was determined for each individual in a combined analysis of protective and susceptibility polymorphisms associated with each disease. Each susceptibility SNP was assigned a value of +1, and each protective SNP was assigned a value of −1. Values were added to derive a net SNP score for each test.

The distribution was examined in terms of the frequency of smokers in respect of each of the 3 tests (Table 14) and in terms of a combined SNP score from adding the SNP scores for each of the three tests (FIG. 23). In Table 14, “3 tests” represents each of the Emphagene™-brand pulmonary test (as described in Example 1 herein), the Bronchogene™-brand lung cancer test (as described in Example 2 herein), and the Cardiogene™-brand cardiovascular test (as described in Example 3 herein), while “2 tests” and “1 test” represent two or one of these tests, respectively.

TABLE 14
Frequency of smokers for each test

| Risk group | 3 tests | 2 tests | 1 test | No tests |
|---|---|---|---|---|
| Low risk | 2 | 21 | 68 | 53 |
| (n = 144) | (1.4%) | (14.6%) | (47.2%) | (36.8%) |
| High risk | 0 | 29 | 63 | 52 |
| (n = 144) | (0%) | (20.1%) | (43.8%) | (36.1%) |

Low risk smokers (combined score −5 to 0) made up 28% (40/144) and high risk smokers (combined score of 5 to 11) made up 24% (36/144) (FIG. 25). As shown above in Table 14, when smokers were divided in to low and high risk for each test and then compared across all 3 tests, for low risk smokers 37% were low risk for all 3 tests, while 16% were low for 2 or 3 tests. For high risk smokers, 20% are high risk for 2+ tests while 36% are not high risk for any of the 3 tests.

When the SNP scores for each of the three tests were added to together, a combined SNP score was derived. A normal distribution of combined score amongst the smokers was observed (see FIG. 23).

This normal distribution of combined scores provides a powerful overall tool for risk assessment, particularly in determining whether a given subject is suitable for an intervention as described herein.

It is not the intention to limit the scope of the invention to the abovementioned examples only. As would be appreciated by a skilled person in the art, many variations are possible without departing from the scope of the invention as set out in the following indicative claims.

### INDUSTRIAL APPLICATION

The present invention is directed to methods for assessing a subject's suitability for an intervention in respect of a disease. The methods comprise the analysis of polymorphisms herein shown to be associated with increased or decreased risk of developing a disease, or the analysis of results obtained from such an analysis, the determination of a net risk score, and a comparison with a distribution of net risk scores for the disease. Methods of treating subjects at risk of developing a disease herein described are also provided.

### PUBLICATIONS

1. Sandford A J, et al., 1999. Z and S mutations of the α1-antitrypsin gene and the risk of chronic obstructive pulmonary disease. Am J Respir Cell Mol Biol. 20; 287-291.
2. Maniatis, T., Fritsch, E. F. and Sambrook, J., Molecular Cloning Manual. 1989.
3. Papafili A, et al., 2002. Common promoter variant in cyclooxygenase-2 represses gene expression. Arterioscler Thromb Vasc Biol. 20; 1631-1635.
4. Ukkola, O., Erkkilä, P. H., Savolainen, M. J. & Kesäniemi, Y. A. 2001. Lack of association between polymorphisms of catalase, copper zinc superoxide dismutase (SOD), extracellular SOD and endothelial nitric oxide synthase genes and macroangiopathy in patients with type 2 diabetes mellitus. J Int Med 249; 451-459.
5. Smith C A D & Harrison D J, 1997. Association between polymorphism in gene for microsomal epoxide hydrolase and susceptibility to emphysema. Lancet. 350; 630-633.
6. Lorenz E, et al., 2001. Determination of the TLR4 genotype using allele-specific PRC. Biotechniques. 31; 22-24.
7. Cantlay A M, Smith C A, Wallace W A, Yap P L, Lamb D, Harrison D J. Heterogeneous expression and polymorphic genotype of glutathione S-transferases in human lung. Thorax. 1994, 49 (10): 1010-4.
8. Wald N J, et al., 1999. When can a risk factor be used as a worthwhile screening test? BMJ 319:1562-1565.
9. Reich D E et al., 2001. Linkage disequilibrium in the human genome, Nature. 411:199-204
10. Leigh et al. Chest 2002, 121, 264.
11. Hnizdo et al. Am J Epidemiol. 2002, 156, 738.
12. Sandford A J, et al., 1999. Z and S mutations of the α1-antitrypsin gene and the risk of chronic obstructive pulmonary disease. Am J Respir Cell Mol Biol. 20; 287-291.
13. Alberg A J, Samet J M. Epidemiology of lung cancer. Chest 2003, 123, 21s-49s.
14. Schwartz A G. Genetic predisposition to lung cancer. Chest 2004, 125, 86s-89s.
15. Wu X, Zhao H, Suk R, Christiani D C. Genetic susceptibility to tobacco-related cancer. Oncogene 2004, 23, 6500-6523.
16. Anthonisen N R. Prognosis in COPD: results from multi-center clinical trials. Am Rev Respir Dis 1989, 140, s95-s99.
17. Skillrud D M, et al. Higher risk of lung cancer in COPD: a prospective matched controlled study. Ann Int Med 1986, 105, 503-507.
18. Tockman M S, et al. Airways obstruction and the risk for lung cancer. Ann Int Med 1987, 106, 512-518.
19. Kuller L H, et al. Relation of forced expiratory volume in one second to lung cancer mortality in the MRFIT. Am J Epidmiol 1190, 132, 265-274.
20. Nomura a, et al. Prospective study of pulmonary function and lung cancer. Am Rev Respir Dis 1991, 144, 307-311.
21. Canne S T, et al. Previous lung disease and risk of lung cancer among men and women nonsmokers. Am J Epidemiol 1999, 149, 13-20.

All patents, publications, scientific articles, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of various embodiments or preferred embodiments and are exemplary only and not intended as limitations on the scope of the invention. Other objects, aspects, examples and embodiments will occur to those skilled in the art upon consideration of this specification, and are encompassed within the spirit of the invention as defined by the scope of the claims. It will be readily apparent to one skilled in the art that varying substitutions and modifications can be made to the invention disclosed herein without departing from the scope and spirit of the invention. The invention illustratively described herein suitably can be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” can be replaced with either of the other two terms in the specification, thus indicating additional examples, having different scope, of various alternative embodiments of the invention. Also, the terms “comprising”, “including”, containing”, etc. are to be read expansively and without limitation. The methods and processes illustratively described herein suitably can be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances can the patent be interpreted to be limited to the specific examples or embodiments or methods specifically disclosed herein. Under no circumstances can the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed can be resorted to by those skilled in the art, and that such modifications and variations are considered to be within the scope of this invention.

1. A method of assessing a subject's suitability for an intervention that is diagnostic of or therapeutic for a disease, the method comprising:
a) providing a net score for said subject, wherein the net score is or has been determined by:
i) providing the result of one or more genetic tests of a sample from the subject, and analysing the result for the presence or absence of protective polymorphisms and for the presence or absence of susceptibility polymorphisms, wherein said protective and susceptibility polymorphisms are associated with said disease,
ii) assigning a positive score for each protective polymorphism and a negative score for each susceptibility polymorphism or vice versa;
iii) calculating a net score for said subject by representing the balance between the combined value of the protective polymorphisms and the combined value of the susceptibility polymorphisms present in the subject sample; and
b) providing a distribution of net scores for disease sufferers and non-sufferers wherein the net scores for disease sufferers and non-sufferers are or have been determined in the same manner as the net score determined for said subject;
c) determining whether the net score for said subject lies within a threshold on said distribution separating individuals deemed suitable for said intervention from those for whom said intervention is deemed unsuitable;
wherein a net score within said threshold is indicative of the subject's suitability for the intervention, and wherein a net score outside the threshold is indicative of the subject's unsuitability for the intervention.

2. The method according to claim 1, wherein the value assigned to each protective polymorphism is the same.

3. The method according to claim 1, wherein the value assigned to each susceptibility polymorphism is the same.

4. The method according to claim 1, wherein each protective polymorphism has a negative value and each susceptibility polymorphism has a positive value.

5. The method according to claim 1, wherein each protective polymorphism has a positive value and each susceptibility polymorphism has a negative value.

6. The method according to claim 1, wherein when the disease is a lung disease, the protective polymorphisms analysed may be selected from one or more of the group consisting of:
+760GG or +760CG within the gene encoding superoxide dismutase 3 (SOD3);
−1296TT within the promoter of the gene encoding tissue inhibitor of metalloproteinase 3 (TIMP3);
CC (homozygous P allele) within codon 10 of the gene encoding transforming growth factor beta (TGFβ);
2G2G within the promoter of the gene encoding metalloproteinase 1 (MMP1); or one or more polymorphisms in linkage disequilibrium with one or more of these polymorphisms.

7. The method according to claim 6, wherein all polymorphisms of the group are analysed.

8. The method according to claim 1, wherein when the disease is a lung disease, the susceptibility polymorphisms analysed are selected from one or more of the group consisting of:
−82AA within the promoter of the gene encoding human macrophage elastase (MMP12);
−1562CT or −1562TT within the promoter of the gene encoding metalloproteinase 9 (MMP9);
1237AG or 1237AA (Tt or tt allele genotypes) within the 3′ region of the gene encoding α1-antitrypsin (α1AT); or
one or more polymorphisms in linkage disequilibrium with one or more of these polymorphisms.

9. The method according to claim 8, wherein all polymorphisms of the group are analysed.

10. The method according to claim 1, wherein when the disease is COPD, the protective polymorphisms analysed may be selected from one or more of the group consisting of:
−765 CC or CG in the promoter of the gene encoding cyclooxygenase 2 (COX2);
Arg 130 Gln AA in the gene encoding Interleukin-13 (IL-13);
Asp 298 Glu TT in the gene encoding nitric oxide synthase 3 (NOS3);
Lys 420 Thr AA or AC in the gene encoding vitamin binding protein (VDBP);
Glu 416 Asp TT or TG in the gene encoding VDBP;
Ile 105 Val AA in the gene encoding glutathione S-transferase (GSTP1);
MS in the gene encoding α1-antitrypsin (α1AT);
the +489 GC genotype in the gene encoding Tissue Necrosis factor α (TNFα);
the −308 GG genotype in the gene encoding TNFα;
the C89Y AA or AG genotype in the gene encoding SMAD3;
the 161 GG genotype in the gene encoding Mannose binding lectin 2 (MBL2);
the −1903 AA genotype in the gene encoding Chymase 1 (CMA1);
the Arg 197 Gln AA genotype in the gene encoding N-Acetyl transferase 2 (NAT2);
the His 139 Arg GG genotype in the gene encoding Microsomal epoxide hydrolase (MEH);
the −366 AA or AG genotype in the gene encoding 5 Lipo-oxygenase (ALOX5);
the HOM T2437C TT genotype in the gene encoding Heat Shock Protein 70 (HSP 70);
the exon 1+49 CT or TT genotype in the gene encoding Elafin;
the Gln 27 Glu GG genotype in the gene encoding β2 Adrenergic receptor (ADBR);
the −1607 1 G1G or 1G2G genotype in the promoter of the gene encoding Matrix Metalloproteinase 1 (MMP1);
or one or more polymorphisms in linkage disequilibrium with one or more of these polymorphisms.

11. The method according to claim 10, wherein all polymorphisms of the group are analysed.

12. The method according to claim 1, wherein when the disease is COPD, the susceptibility polymorphisms analysed are selected from one or more of the group consisting of:
Arg 16 Gly GG in the gene encoding β2-adrenoreceptor (ADRB2);
105 AA in the gene encoding Interleukin-18 (IL-18);
−133 CC in the promoter of the gene encoding IL-18;
−675 5G5G in the promoter of the gene encoding plasminogen activator inhibitor
−1055 TT in the promoter of the gene encoding IL-3;
874 TT in the gene encoding interferon gamma (IFNγ);
the +489 AA or AG genotype in the gene encoding TNFα;
the −308 AA or AG genotype in the gene encoding TNFα;
the C89Y GG genotype in the gene encoding SMAD3;
the E469K GG genotype in the gene encoding Intracellular Adhesion molecule 1 (ICAM1);
the Gly 881 Arg GC or CC genotype in the gene encoding Caspase (NOD2);
the −511 GG genotype in the gene encoding IL1B;
the Tyr 113 His TT genotype in the gene encoding MEH;
the −366 GG genotype in the gene encoding ALOX5;
the HOM T2437C CC or CT genotype in the gene encoding HSP 70;
the +13924 AA genotype in the gene encoding Chloride Channel Calcium-activated 1 (CLCA1);
the −159 CC genotype in the gene encoding Monocyte differentiation antigen CD-14 (CD)-14);
or one or more polymorphisms in linkage disequilibrium with one or more of these polymorphisms.

13. The method according to claim 12, wherein all polymorphisms of the group are analysed.

14. The method according to claim 1, wherein when the disease is OCOPD, the protective polymorphisms analysed may be selected from one or more of the group consisting of:
−765 CC or CG in the promoter of the gene encoding COX2:
−251 AA in the promoter of the gene encoding interleukin-8 (IL-8);
Lys 420 Thr AA in the gene encoding VDBP;
Glu 416 Asp TT or TG in the gene encoding VDBP;
exon 3 T/C RR in the gene encoding microsomal epoxide hydrolase (MEH);
Arg 312 Gln AG or GG in the gene encoding SOD3;
MS or SS in the gene encoding α1AT;
Asp 299 Gly AG or GG in the gene encoding toll-like receptor 4 (TLR4);
Gln 27 Glu CC in the gene encoding ADRB2;
−518 AA in the gene encoding IL-11;
Asp 298 Glu TT in the gene encoding NOS3; or
one or more polymorphisms in linkage disequilibrium with one or more of these polymorphisms.

15. The method according to claim 14, wherein all polymorphisms of the group are analysed.

16. The method according to claim 1, wherein when the disease is OCOPD, the susceptibility polymorphisms analysed are selected from one or more of the group consisting of:
−765 GG in the promoter of the gene encoding COX2;
105 AA in the gene encoding IL-18;
−133 CC in the promoter of the gene encoding IL-18;
−675 5G5G in the promoter of the gene encoding PAI-1;
Lys 420 Thr CC in the gene encoding VDBP;
Glu 416 Asp GG in the gene encoding VDBP;
Ile 105 Val GG in the gene encoding GSTP1;
Arg 312 Gln AA in the gene encoding SOD3;
−1055 TT in the promoter of the gene encoding IL-13;
3′ 1237 Tt or tt in the gene encoding α1AT;
−1607 2G2G in the promoter of the gene encoding MMP1; or
one or more polymorphisms in linkage disequilibrium with one or more of these polymorphisms.

17. The method according to claim 16, wherein all polymorphisms of the group are analysed.

18. The method according to claim 1, wherein when the disease is lung cancer, the protective polymorphisms analysed may be selected from one or more of the group consisting of:
the Asp 298 Glu TT genotype in the gene encoding NOS3;
the Arg 312 Gln CG or GG genotype in the gene encoding SOD3;
the Asn 357 Ser AG or GG genotype in the gene encoding MMP12;
the 105 AC or CC genotype in the gene encoding IL-18;
the −133 CG or GG genotype in the gene encoding IL-18;
the −765 CC or CC genotype in the promoter of the gene encoding COX2;
the −221 TT genotype in the gene encoding Mucin 5AC (MUC5AC);
the intron 1 C/T TT genotype in the gene encoding Arginase 1 (Arg1);
the Leu252Val GG genotype in the gene encoding Insulin-like growth factor II receptor (IGF2R);
the −1082 GG genotype in the gene encoding Interleukin 10 (IL-10);
the −251 AA genotype in the gene encoding Interleukin 8 (IL-8);
the Arg 399 Gln AA genotype in the X-ray repair complementing defective in Chinese hamster 1 (XRCC1) gene;
the A870G GG genotype in the gene encoding cyclin D (CCND1);
the −751 GG genotype in the promoter of the xeroderma pigmentosum complementation group D (XPD) gene;
the Ile 462 Val AG or GG genotype in the gene encoding cytochrome P450 1A1 (CYP1A1)
the Ser 326 Cys GG genotype in the gene encoding 8-Oxoguanine DNA glycolase (OGG1);
the Phe 257 Ser CC genotype in the gene encoding REV1;
the E375G T/C TT genotype in the gene encoding CAMKK1;
the −81 C/T (rs2273953) CC genotype the gene encoding TP73;
the A/C (rs2279115) AA genotype in the gene encoding BCL2;
the +3100 A/G (rs2317676) AG or GG genotype in the gene encoding ITGB3;
the C/Del (rs1799732) CDel or DelDel genotype in the gene encoding DRD2; or
the C/T (rs763110) TT genotype in the gene encoding FasL;
or one or more polymorphisms in linkage disequilibrium with any one or more of these polymorphisms.

19. The method according to claim 18, wherein all polymorphisms of the group are analysed.

20. The method according to claim 1, wherein when the disease is lung cancer, the susceptibility polymorphisms analysed are selected from one or more of the group consisting of:
the −786 TT genotype in the promoter of the gene encoding NOS3;
the Ala 15 Thr GG genotype in the gene encoding anti-chymotrypsin (ACT);
the 105 AA genotype in the gene encoding IL-18;
the −133 CC genotype in the promoter of the gene encoding IL-18;
the 874 AA genotype in the gene encoding IFNγ;
the −765 GG genotype in the promoter of the gene encoding COX2;
the −447 CC or GC genotype in the gene encoding Connective tissue growth factor (CTGF); and
the +161 AA or AG genotype in the gene encoding MBL2.
the −511 GG genotype in the gene encoding IL-1B;
the A-670G AA genotype in the gene encoding FAS (Apo-1/CD95);
the Arg 197 Gln GG genotype in the gene encoding N-acetyltransferase 2 (NAT2);
the Ile 462 Val AA genotype in the gene encoding CYP1A1;
the 1019 G/C Pst I CC or CG genotype in the gene encoding cytochrome P450 2E1 (CYP2E1);
the C/T Rsa I TT or TC genotype in the gene encoding CYP2E1;
the GSTM null genotype in the gene encoding GSTM;
the −1607 2G/2G genotype in the promoter of the gene encoding MMP1;
the Gln 185 Glu CC genotype in the gene encoding Nibrin (NBS1);
the Asp 148 Glu GG genotype in the gene encoding Apex nuclease (APE1);
the R19W A/G AA or GG genotype in the gene encoding Cer 1;
the Ser 307Ser G/T GG or GT genotype in the XRCC4 gene;
the K3326X A/T AT or TT genotype in the BRCA2 gene;
the V433M A/G AA genotype in the gene encoding Integrin alpha-11;
the A/T c74delA AT or TT genotype in the gene encoding CYP3A43;
the −3714 G/T (rs6413429) GT or TT genotype in the gene encoding DAT1;
the A/G (rs1139417) AA genotype in the gene encoding TNFR1; or
the C/T (rs5743836) CC genotype in the gene encoding TLR9;
or one or more polymorphisms in linkage disequilibrium with any one or more of these polymorphisms.

21. The method according to claim 20, wherein all polymorphisms of the group are analysed.

22. The method according to claim 1, wherein each protective polymorphism is assigned a value of −1 and each susceptibility polymorphism is assigned a value of +1.

23. The method according to claim 1, wherein each protective polymorphism is assigned a value of +1 and each susceptibility polymorphism is assigned a value of −1.

24. The method according to claim 1, wherein the subject is or has been a smoker.

25. The method according to claim 1, wherein the method comprises an analysis of one or more risk factors, including one or more epidemiological risk factors, associated with the risk of developing said disease.

26. A method for the diagnostic, prophylactic or therapeutic treatment of a disease in a subject whose suitability for said treatment is or has been determined by a method according to claim 1, further comprising the steps of communicating to said subject said net susceptibility score, and advising on changes to the subject's lifestyle that could reduce the risk of developing said disease.

27. A method of assessing a subject's risk of developing two or more diseases, the method comprising the steps of
providing a net score for the subject in respect of each of the two or more diseases; wherein each net score is or has been determined by:
i) providing the result of one or more genetic tests of a sample from the subject, and analysing the result for the presence or absence of protective polymorphisms and for the presence or absence of susceptibility polymorphisms, wherein said protective and susceptibility polymorphisms are associated with at least one of the two or more diseases,
ii) assigning a positive score for each protective polymorphism and a negative score for each susceptibility polymorphism or vice versa;
iii) calculating a net score for said subject by representing the balance between the combined value of the protective polymorphisms and the combined value of the susceptibility polymorphisms present in the subject sample;
and
combining the two or more net scores to give a combined score, said combined score representing the balance between the combined value of the subject's protective polymorphisms and the combined value of the subject's susceptibility polymorphisms for each of the two or more diseases;
wherein a combined protective score is predictive of a reduced risk of developing the two or more diseases and a combined susceptibility score is predictive of an increased risk of developing the two or more diseases.

28. The method according to claim 27, wherein the two or more diseases are selected from the group comprising COPD, OCOPD, lung cancer, or ACS.

29. The method according to claim 28, wherein the two or more diseases are COPD, lung cancer and ACS.

30. (canceled)

31. The method according to claim 37, wherein the two or more diseases are selected from the group comprising COPD, OCOPD, lung cancer, or ACS.

32. The method according to claim 31, wherein the two or more diseases are COPD, lung cancer and ACS.

33. (canceled)

34. (canceled)

35. A kit for assessing a subject's suitability for an intervention diagnostic of or therapeutic for a disease, said kit comprising a means of analysing a sample from said subject for the presence or absence of one or more protective polymorphisms and one or more susceptibility polymorphisms in accordance with a method of claim 1.

36. A kit for assessing a subject's suitability for an intervention diagnostic of or therapeutic for a disease, said kit comprising a means of analysing a sample from said subject for the presence or absence of one or more protective polymorphisms and one or more susceptibility polymorphisms in accordance with a method of claim 27.

37. A method of assessing a subject's risk of developing two or more diseases, comprising evaluating a combined score for the subject, wherein the combined score represents the balance between a combined value of the subject's protective polymorphisms and a combined value of the subject's susceptibility polymorphisms for each of the two or more diseases, and wherein a combined protective score is predictive of a reduced risk of developing the two or more diseases and a combined susceptibility score is predictive of an increased risk of developing the two or more diseases.
